SEQUENCE LISTING (20 sequences)

SEQ ID NO: 1 - DNA, 18 bp, Artificial sequence
  Description of the artificial sequence Primer EGFPwt-1
gtgaccaccctgacctac

SEQ ID NO: 2 - DNA, 20 bp, Artificial sequence
  Description of the artificial sequence Primer EGFPSeq1
gcagcacgacttcttcaagt

SEQ ID NO: 3 - DNA, 21 bp, Artificial sequence
  Description of the artificial sequence Primer EGFPSeq3
gttgtggctgttgtagttgta

SEQ ID NO: 4 - DNA, 73 bp, Artificial sequence
  Description of the artificial sequence Primer EGFP-11
gttcatctgcaccaccggcaagctgcccggaattctagggataacagggtaatgcttcagccgctaccccgac

SEQ ID NO: 5 - DNA, 41 bp, Artificial sequence
  Description of the artificial sequence Primer EGFP-13
aggagggaattcggatccgcggccgctttacttgtacagct

SEQ ID NO: 6 - DNA, 33 bp, Artificial sequence
  Description of the artificial sequence Primer EGFP-14
tccctagaattccgggcagcttgccggtggtgc

SEQ ID NO: 7 - DNA, 58 bp, Artificial sequence
  Description of the artificial sequence Primer EGFP-15
cgcgcgacctggtgcatgacccgcaagcccggtgccatggtgagcaagggcgaggagc

SEQ ID NO: 8 - DNA, 37 bp, Artificial sequence
  Description of the artificial sequence Primer EGFP-EJ-1
tgacctactagggataacagggtaatcctacggcgtg

SEQ ID NO: 9 - DNA, 49 bp, Artificial sequence
  Description of the artificial sequence Primer EGFP-EJ-2B
cacgccgtaggattaccctgttatccctagtaggtcagggtggtcacga

SEQ ID NO: 10 - DNA, 20 bp, Artificial sequence
  Description of the artificial sequence Primer Puro1
cccgcaacctccccttctac

SEQ ID NO: 11 - DNA, 21 bp, Artificial sequence
  Description of the artificial sequence Primer EGFP20-2
gtgaacagctcctcgcccttg

SEQ ID NO: 12 - DNA, 22 bp, Artificial sequence
  Description of the artificial sequence Primer Sce1-1
ggtccgaactctaaactgctga

SEQ ID NO: 13 - DNA, 20 bp, Artificial sequence
  Description of the artificial sequence Primer Sce1-2b
tcggggtcaggtagttttca

SEQ ID NO: 14 - DNA, 20 bp, Artificial sequence
  Description of the artificial sequence Primer EGFP-PCR1
gtgagcaagggcgaggagct

SEQ ID NO: 15 - DNA, 23 bp, Artificial sequence
  Description of the artificial sequence Primer EGFP-PCR2
ctttacttgtacagctcgtccat

SEQ ID NO: 16 - DNA, 24 bp, Artificial sequence
  Description of the artificial sequence Primer EGFP-PCR3
gacgttgtggctgttgtagttgta

SEQ ID NO: 17 - DNA, 22 bp, Artificial sequence
  Description of the artificial sequence Primer Hyg1-1
ggaaagcctgaactcaccgcga

SEQ ID NO: 18 - DNA, 22 bp, Artificial sequence
  Description of the artificial sequence Primer Hyg2-2
gcttctgcgggcgatttgtgta

SEQ ID NO: 19 - DNA, 22 bp, Artificial sequence
  Description of the artificial sequence Primer Hyg4-1
tacacaaatcgcccgcagaagc

SEQ ID NO: 20 - DNA, 9320 bp, Artificial sequence
  Description of the artificial sequence Plasmid p5-Puro-CMV-(N′-EGFP)-CMV-Red-(EGFP-EJ)
cgattagtccaatttgttaaagacaggatatcaggtggtccaggctctagttttgactcaacaatatcaccagctgaagcctatagagtacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgattgactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagaccccagcccagggaccaccgacccccccgccgggaggcaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctaggagacgtcccagcggcctcgggggcccgttttgtggcccgttctgtgtcgttaaccacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagggccagactgttaccactcccttaagtttgaccttagatcactggaaagatgtcgagcggatcgctcacaaccagtcggtagatgtcaagaagagacgatgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctctttcgaccccgcctcgatcctccctttatccagccctcactccttctctaggcggctccaccgcggtggcggccgctctagaactagtggatccggggccggatcagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagctcgatatcaagcttcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagagcttatcgataccgtcgaggccgcggatcccccgggctgcaggaattcgagctcggtaccctaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagcggcctcgagctcaagctcctggtgcatgacccgcaagcccggtgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctactagggataacagggtaatcctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctcgagggggggcccgcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctagcttaagtaagccattttgcaaggcatggaaaaatacataactgagaatagagaagttcagatcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactacccgtcagcgggggtctttcagtttctcccacctacacaggtctcactaacattcctgatgtgccgcagggactccgtcagcccggtttgtgtttataataaaatgcaagaacagtgttcccttcaagccagactacatcctgactctcggctttataaaagaatgttgaagggctctgtggactatctgccacacgacttttaagatttttatgcctcctggatgagggatttagtcaatctatcctcgtctattttgctggcttctccgtattttaaatttctagtttgcactcccttcctgagagcacggcgattgcagagtagttaatactctgagggcaggcttctgtgaaaaggttgcctgggctcagtgtgagattttgccataaaaaggggtcctgcccctgtgtacagacagatcggaatctagagtgcatactcagagtccccgcggttccggggctctgatctcagggcatctttgcctagagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagg

(19) United States
(12) Patent Application Publication
(10) Pub. No.: US 2004/0197855 A1
(43) Pub. Date: Oct. 7, 2004

(54) Test system for determining gene toxicities

(76) Inventor: Lisa Wiesmuller, Augsburg (DE)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/471,065

(22) PCT Filed: Feb. 28, 2002
(86) PCT No.: PCT/EP02/02194

(30) Foreign Application Priority Data
Mar. 5, 2001 (DE) 101 10 449.9

(51) Int. Cl. C07H 21/04; C12N 15/00; C07K 14/435

(52) U.S. Cl. 435/69.1; 435/320.1; 435/325; 530/350; 536/23.5

(57) ABSTRACT

The subject matter of the invention is a method which makes it possible to detect genotoxic stress in living mammalian cells by the detection of all previously documented types of DNA recombination and also of mutations in response to noxae. The detection is based on the detection of the fluorescence from intact autofluorescent proteins or of luminescence by means of intact bioluminescent enzymes or enzymes that convert chemoluminescent substrates. It is characterised by a short reaction time in the scope of hours and permits the transfer of the test system to different mammalian cells and to living experimental animals, particularly by using a retroviral vector system.

### 1. SUBJECT MATTER OF THE INVENTION

The subject matter of the invention is a method which makes the detection of genotoxic stress in living mammalian cells possible by the detection of all previously documented types of DNA recombination and also of mutations in response to noxae. The detection is based on the detection of the fluorescence from intact autofluorescent proteins or of luminescence by means of intact bioluminescent enzymes or enzymes that convert chemoluminescent substrates, it is characterised by a short reaction time in the scope of hours and permits the transfer of the test system to different mammalian cells and to living experimental animals, particularly by using a retroviral vector system in particular.

### 2. STATE OF THE ART

Excluding possible genotoxicities is one of the basic preconditions for the official approval of new food constituents, cosmetic products, and medicaments. The existing processes for identifying genotoxicities can be subdivided on the basis of the determination of different biological responses to treatment with a genotoxic agent as follows: In animal experiments, physiological parameters such as changes in body weight, water consumption, the blood picture, blood chemistry or histological changes are detected, in addition to the formation of tumours (Burdock, G. A. et al. (2000) Food Chem. Toxicol. 38: 429-442). At the molecular level, methods for detecting genetic changes have been successful in the main. Major chromosome rearrangements are thus cytogenetically assessed as an increase in sister chromatid exchanges (SCE) in human or murine bone marrow cells (Hadagny, W. et al. (1989) Mutat. Res. 225: 27-32; Burdock, G. A. et al. (2000) Food Chem. Toxicol. 38: 429-442), the formation of micronuclei is observed in the lymphocytes of exposed persons or in the cells of indicator organisms, e.g. the plant Tradescantia (Kazimierz, G. and Brokos, B. (2000) Mutagenesis 15: 137-141; Monarca, S. et al. (1999) Mutat. Res. 426: 189-192), mutations in the HPRT gene are measured via the outgrowth of mammalian cell clones resistant to 6-thioguanine following the exposure to harmful substances (O' Donovan, M. R. et al. (1990) Mutagenesis 5: 275-277), or genetic changes in the DNA of shuttle vectors are analysed by phenotypically detecting the mutations after transfer of the vector DNA from the test organism into bacteria (de Groene, E. M. et al. (1995) Eur. J. Pharmacol. 293: 47-53). Moreover, the detection of unrepaired DNA strand breaks in lymphocyte DNA by way of the so-called Comet assay (Somorovska, M. et al. (1999) Mutat. Res. 445: 181-192) and the detection of increased DNA repair synthesis activities in primary rat liver cells (Williams, G. M. (1977) Cancer. Res. 37: 1845-1851) are available as indicators of previous DNA damage.

The meaningfulness of the above-mentioned physiological investigations in the living animal regarding the carcinogenicity of certain substances is limited, the detection of tumours actually formed following primary damage is highly insensitive since the formation of a tumour is preceded by the alteration of thousands of genes (Loeb, L. and Loeb, L. A. (2000) Carcinogenesis 21: 379-385). Each one of the molecular methods described above detects damage to the genome which has arisen in cells of the eukaryotic target organism and, during the assessment of potentially genotoxic substances, any possible metabolic activation of mutagenic effects is consequently taken into consideration. Nevertheless, these methods have certain limitations: the detection of SCEs or of micronuclei requires the preparation, fixation, and staining of the cell material, and subsequent examination under the microscope. Moreover, for SCE detection, treatment with colchicine or bromodeoxyuridine is additionally required before sample preparation, for the micronucleus assay, the treatment involves cytochalasin B, whose effects on the treatment with the compounds under investigation have not yet been clarified. The detection of 6-thioguanine resistance in eukaryotic cells requires either a time consuming cultivation procedure during which the original effect of the toxic agent may be modified or microscopic analyses following treatment with bromodeoxyuridine. Working with shuttle vectors requires the recovery of the vector DNA from the eukaryotic cells and their reintroduction into bacteria. During the latter, mostly chemical process, the vector DNA is increasingly attacked by bacterial, DNA modifying enzymes, and this can lead to an apparent increase in the mutagenicity of the agent originally analysed in mammalian cells. The same effect can be caused by subtle physiological disturbances in the bacteria used, since the bacterial mutation rate is 1-2 orders of magnitude higher than that of eukaryotic cells, even without any exogenous influence (Friedberg, E. C. (1984) in DNA Repair, W. H. Freeman and Company, New York). The greatest difficulties with the Comet assay and with the quantification of DNA repair synthesis activities seem to be the large variations of the data, so that numerous multiple determinations need to be done, as far as possible with automated data logging, to obtain statistically relevant data. In addition, the determination of DNA repair synthesis activities for detection purposes is mostly preceded by treatment with radioactively labelled nucleotide precursors, again with unclear effects on the effects of the noxae of interest.

In some cases, changes in the gene expression pattern have been used for visualising the cellular response to DNA damage in eukaryotes (Billinton, N. et al. (1998) Biosensors & Bioelectronics 13: 831-838; Todd, M. D. et al. (1999) J. Biomol. Screen 4: 259-268). Gene expression profiling is in general a method which may well gain increasing importance in connection with the chip array technique. However, the need, usually connected with this method, for carrying out nucleic acid and/or protein extractions has to be mentioned as a disadvantage thereof. It increases not only the amount of labour and time that have to be invested for the detection process, but also harbours the risk of a lack of reproducibility. In the case of one process which is based on the detection of Rad54 gene activation inducible by DNA damage, the nucleic acid/protein extraction step was rendered unnecessary by coupling of the inducible Rad54 promoter to an EGFP reporter cDNA (Billinton, N. et al. (1998) Biosensors & Bioelectronics 13: 831-838). Here, DNA damage was indicated by the green fluorescence emitted by the gene product EGFP (enhanced green fluorescent protein). Following excitation of the EGFP autofluorescence by light at a wavelength of 488 nm, it was possible to determine the light emission at a wavelength of 511 nm fluorometrically. Critical disadvantages of this method are that, firstly, it was established for Saccharomyces cerevisiae rather than for mammalian cells and, secondly, fluorescence can be measured only after a series of extraction steps, i.e. not with intact cells.

The genotoxicity assay by far the most frequently used is the Ames assay (Ames, B. N. et al. (1973) Proc. Natl. Acad. Sci. USA 70: 2281-2285; Maron, D. M. and Ames, B. N. (1983) Mutat. Res. 113: 173-215). This assay is used in the pharmaceutical industry in order to allow preselections during the development of new drugs and to test these drugs during approval procedure. This assay is based on the detection of reverse, i.e. reconstituting, point mutations in the his gene of a defective mutant of Salmonella typhimurium by quantifying the growth in cultures without histidine. Depending on the Ames-Salmonella strain and the noxious substance, the mutations may be of the type of base substitutions, frame shifts, or complex frame shifts with adjacent base substitutions and may be caused by the agent itself or by subsequent erroneous repair processes.

The Ames assay has the critical disadvantage that Salmonella does not possess the cellular machinery of eukaryotic cells which is necessary, to convert procarcinogens into reactive metabolites. For this reason, numerous compounds with a genotoxic effect, e.g. on man, are not detected with this test. This applies also to more recent bacterial assays such as the SOS chromotest (Quillardet, P. et al. (1982) Proc. Natl. Acad. Sci. USA 79: 5971-5975). Although attempts have been made to optimise the Ames assay by adding rat liver extracts to the assay mixture in order to simulate the human metabolism, these extracts do not necessarily contain sufficient enzymatic activities which are necessary for biotransformation (Ames, B. N. (1974) Genetics 78: 91-95). Moreover, the methodology of the Ames assay makes automation as the basis for standardised surveillance procedures more difficult. Furthermore, the majority of all DNA damages are repaired without defects. However, the Ames assay only measures erroneous DNA repair leading to an underestimation of DNA damage. Moreover, larger chromosome rearrangements such as insertions, deletions or translocations are not detected by this assay. Thus, o-toluidine and o-anisidine were classified negative in the Ames assay even though these substances cause tumours in mice and rats (Gold, L. S. et al. (1991) Environ. Health Perspect. 100: 65-168). In contrast, the appearance of strand breaks and increased DNA repair synthesis activities were seen with o-toluidine (Danford N. (1991) Mutat. Res. 258: 207-236). For o-anisidine, indications of genotoxicities could be obtained until last year only on the basis of a slight increase in point mutations in the bladder of the so-called Big Blue indicator mice (see below) (Ashby J. et al. (1991) Carcinogenesis 15: 2291-2296). To sum up, many substances with a mutagenic or carcinogenic effect on man cannot unequivocally be identified as such by using the genotoxicity tests available today. Only 50% of all carcinogens are detected by the Ames assay (Ashby, J. and Tennant, R. W. (1991) Mutat Res. 257: 229-306).

To take into account the fact that larger genome rearrangements than the point mutations investigated in the Ames assay are carcinogenic (Tlsty, T. D. et al. (1995) Mutat Res. 337: 1-7; Sandberg, A. A. (1991) Mutat. Res. 247: 231-240; Bishop, J. M. (1987) Science 235: 305-311; Loeb, L. and Loeb, L. A. (2000) Carcinogenesis 21: 379-385), Schiestl and colleagues (Brennan, R. J. and Schiestl, R. H. (1999) Mutat. Res. 430: 3745) developed assay systems (DEL assay) which are capable of detecting deletion events at the genomic level. Recombination events leading to deletions are triggered in yeast and mammalian cells by numerous carcinogens, such as anilines and o-toluidine, and are undetectable with most of the genotoxicity assays existing (Schiestl, R. H. (1989) Nature 337: 285-288; Brennan, R. J. et al. (1994) Mutat. Res. 308: 159-167; Carls, N. and Schiestl, R. H. (1994) Mutat. Res. 320: 293-303; Aubrecht, J. et al. (1995) Carcinogenesis 16: 2841-2846; Schiestl, R. H. et al. (1997) Proc. Natl. Acad. Sci. USA 94: 4576-4581). The DEL assay, established for the HIS3 locus of the yeast Saccharomyces cerevisiae, suffers from limitations already described for other methodsm, namely that the biotransformation of procarcinogens specific for mammals is not taken into account and survival rates are measured either via cultivation under certain selection conditions or by determining the uptake of a fluorescent substance by use of cell extracts (Brennan, R. J. and Schiestl, R. H. (1999) Mutat. Res. 430: 37-45).

The detection of recombination events, when stimulated by harmful substances in situ, i.e. in the living animal, provides much more interesting possibilities in comparison with the methods mentioned above and, therefore, must be prepared for the routine determinations carried out in the pharmaceutical industry. Only in the animal model, the analysis of genome stability in response to irradiation or genotoxic agents is possible only in the animal model as a function of physiological processes and of the genotype of the animal. Today the targeted modification of genotypes is effected by producing so-called knockouts or transgenic mice, i.e. mice with certain gene defects or additional gene copies, or by crossing such genetically modified mice. By introducing a genotoxicity test into genetically modified mice, new questions could be raised such as that regarding the importance of individual genes for the susceptibility of the animal or of a certain tissue to the genotoxic effect of mutagens. This represents an extremely promising approach for the future since, in the next few years, individual genotypes can be established for single patients as a result of the introduction of the chip array technology in modern hospitals and medical practices. Simultaneously knowing the individual genotype of a cancer patient (healthy and tumour tissue) and the importance of the genes altered in this patient concerning the response to certain noxious agents, as a result of genotoxicity studies in the corresponding knockout or transgenic mice, would allow the development of an individually optimised chemotherapy protocol. Thus, particularly with respect to some tumour suppressor proteins it is known that functional defects promote genomic instabilities or even cause resistance to mutagens, so that under these genetic conditions tumour cells versus healthy tissue would even possess growth advantages. This applies in particular to the tumour suppressor p53 which regulates growth and DNA repair and which, depending on the mutation in the p53 gene, even causes sensitivity or resistance to chemotherapeutic treatments (McGilland, G. and Fisher, D. E. (1999) J. Clin Invest. 104: 223-225; Albrechtsen, N. et al. (1999) Oncogene 18: 7706-7717; Wiesmüller, L. (2000) J. Biomed. Biotech. 1: 7-10).

So far, test systems for the determination of genotoxicities in living animals have been generated which are based on the reconstitution of the β-galactosidase activity that are either available for measurements of the point mutation frequencies in the lacI gene in the Big Blue mouse (Stratagene) or for measurements of reconstituting recombination frequencies in the lacZ gene in male sperm cells of transgenic mice (Akgün, E. et al. (1997) Mol. Cell. Biol. 517: 5559-5570). A major disadvantage of the so-called Big Blue mouse (Stratagene) and of transgenic mice derived from it is that the mutation indicator gene, i.e. the lacI gene which operates as a repressor of the β-galactosidase expression, needs to be transduced after the genotoxic treatment of the mouse, from the genome via the α-phage into bacteria. Only in the bacteria, the activity of the gene product is detected via the chromogenic β-galactosidase substrate X-Gal (Boehringer Mannheim) by a blue stain. However, since the mutation rate in bacteria—as mentioned—and even more so in λ-phage genomes is several orders of magnitude above that in mammalian cells, a basal rate which, from the physiological point of view, is highly unusual for a mammal must be expected (Friedberg, E. C. (1984) in DNA Repair, W. H. Freeman and Company, New York). This could explain the rates of 10⁻⁵ to 10⁻³ per locus measured in the Big Blue mouse. The second test system of Akgün et al (Moynahan, M. E. et al. (1996). Hum. Mol. Genetics. 5: 875-886; Akgün, E. et al. (1997) Mol. Cell. Biol. 517: 5559-5570) was developed to provide an answer to questions regarding the influence of the development of germ cells but not of the effect of a noxious agent. In this case, a chemical (5-chloromethyl fluorescein-di-β-D-galactopyranoside), which is converted into a fluorescent form by β-galactosidase, was used to detect the reconstituted lacZ gene. However, because of the order of the sequence elements, genomic alterations only of a single homologous recombination sub-type, namely gene conversion, can be demonstrated, and this in male germ line cells exclusively.

Finally, the so-called Pink Eyed Unstable (p^{un}) mouse model from Jackson Laboratory (Bar Harbor, Me.) has recently been used to measure major DNA rearrangements following irradiation with x-rays and treatment of the animals with benzo[a]pyrene (Aubrecht, J. et al. (1999) Carcinogenesis 20: 2229-2236). p^{un} mice carry tandem duplications of the p-gene with a length of 70 kilobase pairs in their genome as a result of which the gene is unstable, i.e. predestined for larger rearrangements of the type of homologous recombination. The wild-type gene is expressed in melanocytes and codes for an integral membrane protein which is necessary for the assembly of a high molecular weight complex which causes black pigmentation in the skin of the mice (Rosemblat, S. et al. (1994) Proc. Natl. Acad. Sci. USA 91: m12071-12075). Thus, the deletion of one copy of the duplicated sequences and the concomitant reconstitution of the wild-type gene could be observed on the basis of the appearance of black spots in the grey skin. This method has the disadvantage that the recombinative events are detectable exclusively if they have taken place in the embryonal premelanocytes, so that statistically significant toxicity determinations would require the analysis of a large number of mice treated at the embryonal stage.

It was consequently the task of the present invention to provide an alternative to the Ames test and to develop a simple, reliable test system or test method (assay) by means of which compounds can be examined as to whether they cause gene recombinations and consequently are genotoxic. Preferably, this test system ought to be suitable for determining the type of recombination events.

The task of the invention is achieved by developing a process which, following the insertion of special vectors into the genome of cells, indicates DNA recombination processes in response to genotoxic treatment by the expression of autofluorescent proteins.

### 3. DESCRIPTION OF THE INVENTION

The subject matter of the present invention consists in particular of a vector which exhibits the general structure

5′-[Ins1]-[Prom1]-[Marker1]-[Prom2]-[Ins2]-[Marker2]-3′

wherein

[Ins1] is a sequence segment which codes for a selection marker, for a transcription factor controlled by tetracyclin or estradiol, e.g. the transactivator rtTA reversely controlled by tetracyclin or for the GalER-VP activated by estradiol, for a protein to be analysed with respect to repair, genome stability, genotoxicity, or cancer susceptibility, for an autofluorescent protein (AFP), for a bioluminescent enzyme, or an enzyme that converts chemoluminescent substrates (LE) or a mutant thereof, or which can be altogether absent,

[Prom1] is a promoter or can be altogether absent,

[Marker1] is a sequence segment which is any desired DNA segment with a homology at least approximately 200 bp in length to the sequence segment of [Marker2] or which codes for a derivative or a mutant of an autofluorescent protein (AFP) or an enzyme that converts chemoluminescent substrates (LE),

[Prom2] is a promoter, a spacer, preferably with a length of approximately 1 kb or may be altogether absent,

[Ins2] is a sequence segment which codes for a selection marker, a transcription factor controlled by tetracyclin or estradiol, e.g. the transactivator rtTA reversely controlled by tetracyclin or for the GalER-VP activated by estradiol, for an AFP, LE or a mutant thereof, for I-SceI or for a protein to be analysed with respect to repair, genome stability, genotoxicity, or cancer susceptibility, e.g. the repair surveillance factor p53, or is a spacer, preferably with a length of 1 kb,

[Marker2] is a sequence segment which is any desired DNA segment with a homology of at least approximately 200 bp to the sequence segment of [Marker1] or which codes for a derivative or a mutant of an autofluorescent protein or a bioluminescent enzyme or an enzyme that converts chemoluminescent substrates (AFP or LE),

wherein [Marker1] and [Marker2] are two homologous DNA sequence segments which trigger the alteration of the gene within the sequence segment in [Ins2] by DNA exchange. According to one embodiment of the invention, [Marker1] and [Marker2] are mutant variants of a gene from which the wild-type gene (AFP or LE) is reconstituted by homologous DNA exchange during homologous recombination.

“Homology” in connection with the invention should be understood to mean a perfect homology, i.e. ideally an identity between the corresponding sequence segments or with sequence differences which are present, at distances of not less than approximately 150 bp, within the otherwise identical sequences.

The vectors, according to the invention, which are described here are preferably retroviral vectors.

“Wild-type gene” should be understood in this connection to mean the wild-type gene in the actual (narrower) sense of the word, variants and/or mutants of this gene exhibiting an autofluorescence or bioluminescence activity or an activity converting chemiluminescent substrates being included within the scope of the present invention. This means that two homologous DNA sequence segments are used as [Marker1] and [Marker2] from which the wild-type gene is reconstituted in one embodiment by homologous DNA exchange during homologous recombination such that the autofluorescence or enzyme activity of the gene product is detectable. The vector is moreover characterised in that losses (deletion of [Ins2]) or modifications of [Ins2] occur in the case of non-conservative homologous recombination or replication slippage between [Marker1] and [Marker2]. The vector is moreover characterised in that, in the case of inactivating mutations in [Ins1] or [Ins2], an inactivation of the gene product concerned occurs (loss of the function of the wild-type genes) and in the case of reverse mutations in [Ins1] or [Ins2] the activation of the gene product (reconstitution of the wild-type function) occurs.

For [Ins1], in contrast to [Ins2], those genes, for example, cannot be considered which, like I-SceI, are to be expressed strictly inducibly since, for [Ins1], the 5′LTR promoter is not exchangeable because of the retroviral vector design.

In the case of the vector construct according to the invention, [Ins1] preferably is a sequence segment which contains a nucleic acid sequence coding for a selection marker, such as an antibiotic resistance gene (for example the puromycin or hygromycin resistance gene or a similarly small gene coding for an alternative selection marker), a nucleic acid sequence coding for a protein to be analysed with respect to repair, genome stability, genotoxicity, or cancer susceptibility, for a transcription factor controlled by tetracyclin or estradiol, e.g. the transactivator rtTA reversely controlled by tetracyclin or the GalER-VP activated by estradiol or a nucleic acid coding for an autofluorescent protein (AFP), for a bioluminescent enzyme or an enzyme that converts chemiluminescent substrates (LE), or for a mutant thereof, or may be absent altogether.

Insofar as, within the framework of the present invention, promoters responsive to tetracyclin or estradiol or transcription factors, such as rtTA or GalER-VP are used, numerous further variants thereof exist which are also included in the invention.

The vector in which [Ins1] is a sequence segment containing a nucleic acid sequence coding for an autofluorescent protein, for a bioluminescent enzyme or an enzyme that converts chemiluminescent substrates or for a mutant thereof is characterised in that the gene encoding AFP or LE can be inactivated by mutations in [Ins1]. Insofar as [Ins1] represents an AFP gene or LE gene inactivated by mutation, the reverse mutations in [Ins1] are detectable by the appearance of a fluorescence or chemiluminescence signal of the product of the wild-type gene concerned.

Preferably, [Ins2] is a sequence segment which contains a nucleic acid sequence coding for a selection marker such as an antibiotic resistance gene (for example the puromycin or hygromycin resistance gene or a similarly small gene coding for an alternative selection marker) or an AFP gene (for example the DsRed gene), an LE gene (for example coding for alkaline phosphatase) or a derived mutant gene thereof, a nucleic acid sequence coding for a transcription factor controlled by one of tetracyclin or estradiol (for example the transactivator rtTA reversely controlled by tetracyclin or the GalER-VP inducible by estradiol), or for a protein to be analysed with respect to repair, genome stability, genotoxicity, or cancer susceptibility, or a spacer with a preferred length of 1 kb.

Insofar as the sequence segments [Ins1] and [Ins2] contain nucleic acid sequences coding for AFP genes or LE genes, it is necessary to ensure that the AFP or LE gene exhibit no significant homologies to possible AFP or LE genes in [Marker1] and in [Marker2] and that the gene products in the case of AFP genes exhibit a fluorescence emission spectrum which significantly differs from that of the AFPs encoded by the Marker genes. This means that a loss of [Ins2] following non-conservative homologous recombination between the DNA segments [Marker1] and [Marker2] and inactivating mutations in [Ins2] or [Ins1] are detectable separately by the loss of the fluorescence or luminescence signal of the products of the wild-type gene concerned. Insofar as [Ins1] and [Ins2] represent AFP or LE genes inactivated by mutation, reverse mutations in [Ins2] and/or in [Ins1] are detectable by the appearance of the specific fluorescence or chemiluminescence signal of the products of the wild-type gene concerned.

Insofar as [Ins2] is a sequence segment which contains a nucleic acid sequence coding for an autofluorescent protein or for a bioluminescent enzyme or an enzyme that converts chemoluminescent substrates or for a mutant thereof, this vector is characterised in that, in the case of non-conservative homologous recombination or replication slippage between the sequence segments [Marker1] and [Marker2] or inactivating mutations in [Ins2], this [Ins2] is lost and/or the gene encoding AFP or LE can be inactivated.

Insofar as [Ins2] represents an AFP or LE gene inactivated by mutation, reverse mutations in [Ins2] are also detectable by the appearance of a luminescence or chemiluminescence signal of the product of the wild-type gene concerned.

According to one embodiment of the invention, the sequence segments [[Prom1]-[Marker1]] and [Marker2] can be exchanged in their positions, if, simultaneously, the orientation of the two sequence segments is reversed (i.e. from 5′->3′ to 3′->5′). In the case of vectors which are unsuitable for the detection of non-conservative homologous recombination events (nkHR), it is allowed to effect the exchange without altering the orientation and the orientation of [[Prom1]-[Marker1]] and/or [Marker2], respectively, can be altered without exchange. The resulting sequence then corresponds to 5′-[Ins1]-(3′-[Marker1]-[Prom1]-5′)-[Prom2]-[Ins2]-[Marker2]-3′, 5′-[Ins1]-[Prom1]-[Marker1]-[Prom2]-[Ins2]-(3′-[Marker2]-5′)-3′,5′-[Ins1]-(3′-[Marker1]-[Prom1]-5′)-[Prom2]-[Ins2]-(3′-[Marker2]-5′)-3′ or 5′-[Ins1]-[Marker2]-[Prom2]-[Ins2]-([Prom1]-[Marker1])-3′.

The subject matter of the invention consists, moreover, of a vector which is derived from the above-mentioned vectors by reversing the orientation of the sequence segment [[Prom2]-[Ins2]].

Of course, the changes in the orientation of sequence segments, the use of alternative selection markers, alternative eukaryotic promoters, alternative AFP genes and spacer genes in comparison with those of the preferred embodiments must not lead to interferences with the 5′LTR or the promoters or to the introduction of actual or cryptic splicing signals.

The sequence segment [Prom1] preferably is a CMV promoter or an alternative eukaryotic promoter (such as for example lentiviral promoters) which guarantees an AFP expression which is at least as high in the various cell and tissues types as when the CMV promoter is used. According to one embodiment, [Prom1] can altogether be missing.

The sequence segment [Prom2] preferably is a promoter from the group consisting of an estradiol-responsive (GRE) or a tetracyclin-responsive (TRE) promoter, in particular the Gal4ER-VP-responsive promoter, or an alternative, strictly inducible promoter (such as e.g. ecdyson-responsive promoters), in particular for a gene contained in the sequence segment [Ins2] coding for I-SceI meganuclease (I-SceI), the SV40 promoter or an alternative eukaryotic promoter (such as the TK promoter), in particular when resistance genes are contained in the sequence segment [Ins2], the CMV promoter or an eukaryotic promoter effecting as high an expression in different cell and tissue types as the CMV promoter (such as e.g. lentiviral promoters), for AFP genes contained in the sequence segment [Ins2]. If [Prom2] is a spacer, it preferably has a length of approximately 1 kb (particularly preferably 0.6 kb). Alternatively, [Prom1] can also be missing altogether.

In the case of the vector, the sequence of the wild-type gene (AFP) is the nucleic acid sequence which codes for the humanized renilla green fluorescent protein (hrGFP), for the enhanced green fluorescent protein (EGFP), for the enhanced blue fluorescent protein (EBFP), for the enhanced cyan fluorescent protein (ECFP), for the enhanced yellow fluorescent, or that which codes for the red fluorescent protein (RFP or DsRed), or a nucleic acid sequence which codes for an enzyme whose activity, like that of the alkaline phosphatase, is detectable via a chemoluminescence reaction, including the variants or mutants derived from the above-mentioned sequences.

According to a preferred embodiment, the autofluorescent protein is selected from the group consisting of humanized renilla green fluorescent protein (hrGFP), enhanced green fluorescent protein (EGFP), enhanced blue fluorescent protein (EBFP), enhanced cyan fluorescent protein (ECFP), enhanced yellow fluorescent protein (EYFP), or red fluorescent protein (RFP or DsRed). The bioluminescent protein is e.g. luciferase, the enzyme that converts chemoluminescent substrates is alkaline phosphatase (Roche Diagnostics, Applied Biosystems), peroxidase (Roche Diagnostics, Applied Biosystems), β-galactosidase (Arakawa, H. et al. (1999) Journal of Bioluminescence and Chemiluminescence 13: 349-354), or luciferase (Baldwin, T. O et al. (1999) in Bioluminescence and Chemiluminescence: Perspective for the 21st Century. (Roda, K. A., Pazzagli, M., Kricka, L. J., and Stanley, P. E., eds.) John Wiley & Sons, England pp. 376-379), for example.

Preferably, a vector is made available in which [Marker1] and [Marker2] contain at least one mutation, the position of the mutations being selected such that

a) the section of the sequence between the position in [Marker2], which corresponds to a mutation in [Marker1] and the first mutation in the 3′ direction in [Marker2] or

b) the section of the sequence between the position in [Marker1] which corresponds to a mutation in [Marker2] and the first mutation in the 3′ direction in [Marker1]

comprises at least 150 base pairs.

Another embodiment relates to a vector in the case of which the sequence segment [Marker1] contains the recognition sequence for meganuclease I-SceI from Saccharomyces cerevisiae or is adjacent to it. This vector is characterised in that a double strand break is generated within this recognition sequence in the presence of I-SceI, thus, causing double strand break repair, i.e. not only a homologous recombination but also non-homologous end joining alone or in combination with homologous recombination. The wild-type gene can also be reconstituted by end joining.

Another embodiment relates to a vector in which the sequence segment [Prom1] has been deleted and the sequence segment [Marker1] starts immediately at the 3′ end of the sequence segment [Ins1]. A vector is also included in which the sequence segment [Prom2] has been deleted and the sequence segment [Ins2] starts immediately at the 3′ end of the sequence segment [Marker1].

The invention also relates to vectors derived from the vector, according to the invention, with the general structure

5′-[Ins1]-[Prom1]-[Marker1]-[Prom2]-[Ins2]-[Marker2]-3′

such as, e.g. a vector in which the sequence segment [Marker2] has been deleted or a vector in which [Ins2] is a transcription factor controlled by tetracyclin or estradiol (e.g. rtTA or GalER-VP), wherein the sequence segment [[TRE]-[I-SceI]] or [[GRE]-[I-SceI]] can be inserted in both directions between the 3′ end of sequence segment [Ins2] and the 5′ end of the sequence segment [Marker2], [1-SceI] being the coding sequence for the meganuclease I-SceI from Saccharomyces cerevisiae or the coding sequence for a protein of interest for the analysis of repair, genome stability, genotoxicity, or cancer susceptibility and [TRE] being a promoter (TRE) and [GRE] a promoter (GRE) and the vector being capable of expressing I-SceI or the protein of interest as a function of tetracyclin or estradiol via a promoter (TRE or GRE) which is controlled by a tetracyclin-responsive transcription factor (e.g. rtTA) or by an estradiol-responsive transcription factor (e.g. GalER-VP).

The invention consequently also relates to a vector derived from the above-mentioned vectors in which [Ins2] is a transcription factor controlled by tetracyclin or estradiol, by inserting the sequence segment [TRE]-[Gen] or [GRE]-[Gen] between the 3′ end of the sequence segment [Ins2] and the 5′ end of the sequence segment [Marker2] in the normal orientation or [Gen]-[GRE] in the reverse orientation, wherein

[Gen] is the nucleic acid sequence encoding a protein which plays a role in the analysis of repair, genome stability, genotoxicity, or susceptibility to cancer and [TRE] is a promoter (TRE) and

[GRE] is a promoter (GRE),

the vector being capable of expressing Gen as a function of tetracyclin or estradiol via a promoter (TRE or GRE) which is regulated by a transcription factor responsive to tetracyclin or estradiol.

The invention also relates to a vector derived from the above-mentioned vectors in the case of which the sequence segment [Marker2] has been deleted, wherein [Marker1] additionally contains a recognition sequence for meganuclease I-SceI from Saccharomyces cerevisiae within the sequence segment or adjacent to it. This vector is characterised in that, within this recognition sequence, a double strand break is created in the presence of I-SceI and triggers double strand break repair, i.e. homologous recombination and end joining. The wild-type gene can also be reconstituted by end joining in the absence of [Marker2].

The subject matter of the invention, moreover, consists of retroviral particles which contain a vector according to the invention as well as eukaryotic cells, in particular mammalian cells into which such a vector has been introduced via transfection or via retroviral transduction.

According to one embodiment, the cells are K562 leukemia cells in which the transcription factor Gal4ER-VP is expressed constitutively, Gal4ER-VP permitting a gene expression strictly inducible by β-estradiol via promoters responsive to Gal4ER-VP such that, e.g., the I-SceI meganuclease activity can be “switched on” in a controlled manner. In other words, the promoter responsive to Gal4ER-VP is used as [Prom2] and a nucleic acid sequence coding for I-SceI is used as [Ins2].

By way of the present invention, it is possible to provide, for the first time, a transgenic, non-human mammal, preferably a rodent, whose germ and somatic cells contain a sequence

5′-[Ins1]-[Prom1]-[Marker1]-[Prom2]-[Ins2]-[Marker2]-3′

wherein the sequence segments [Ins1], [Prom1], [Marker1], [Prom2], [Ins2], and [Marker2] have the meaning indicated above. Moreover, mammals are included which, regarding the structure of the sequence 5′-[Ins1]-[Prom1]-[Marker1]-[Prom2]-[Ins2]-[Marker2]-3′, exhibit the variations and changes or modifications explained above in connection with the vectors according to the invention.

By way of the present invention, a method is thus made available for the determination of genotoxicities in which the above-mentioned (eukaryotic) cells or the above-mentioned transgenic non-human mammals are brought into contact with a test compound, wherein an increase or decrease in fluorescent or luminescent cells corresponding to the activity of the wild-type gene product of [Marker1], [Marker2], [Ins1], or [Ins2], in the above-mentioned cells or in the cells of the above-mentioned mammal, as subsequently determined by means of FACS analysis, fluorescence measurement, fluorescence microscopy, laser scanning cytometry, or by a luminescence detection reaction, indicates the genotoxic effect of the test compound.

The loss of a fluorescence or luminescence activity in the individual cells of the population indicates non-conservative DNA exchange events, replication slippage, or inactivating mutations, if [Ins2] of the vector used is the coding sequence of an AFP or LE and, if [Marker1] or [Marker2] are homologous sequences of at least approximately 200 bp. Insofar as [Ins1] and [Ins2] represent AFP or LE genes inactivated by mutation, reverse mutations in [Ins2] or [Ins1] become detectable by the appearance of a fluorescence or chemiluminescence signal of the product of the wild-type gene concerned. In the case of another fluorescence signal produced by the gene products of the wild-type gene of [Ins1] or [Ins2], these differ from those of possible gene products of the wild-type gene of [Marker1] or [Marker2] since homologies to Marker genes and between the Ins genes as well as similar fluorescence spectra of the AFP/LEs are not permitted.

Finally, the present invention provides further possibilities for application. For example, the use of the vector for the determination of cancer susceptibilities by way of the analysis of a patient's blood, skin or biopsy material is included. In this case, the cells obtained from the proband are to be briefly cultivated according to standard methods, in order to introduce the vector according to the invention, in particular the vector in which [Ins2] is a sequence segment which contains a nucleic acid sequence coding for an autofluorescent protein or a bioluminescent enzyme, or an enzyme that converts chemoluminescent substrates or for a mutant thereof, by means of physical or chemical transfection methods or by retroviral infection, and in order to subsequently analyse the transfected cells via FACS analysis, fluorescence microscopy, laser scanning cytometry, fluorescence measurement, or luminescence detection reaction, wherein cells with fluorescence or luminescence corresponding to the activity of the wild-type gene product of [Marker1] and [Marker2] indicate DNA exchange events. The loss of another fluorescence or luminescence activity in individual cells of the population moreover indicates non-conservative DNA exchange events, replication slippage, or mutations, if [Ins2] of the vector used is the coding sequence of an AFP or LE. From the increase in certain caryotypic abnormalities in lymphocytes of breast cancer patients and of their predisposed relatives of the first degree, increased DNA exchange frequencies are expected at least in the cells of these individuals, but possibly also in patients with other types of cancer and their relatives of the first degree (Patel, R. K. et al. (1997) Int. J. Cancer 72: 1-5; Trivedi, A. H., et al. (1998) Breast Cancer Research and Treatment 48: 187-190). Accordingly, depending on the vector structure, increased DNA exchange activities and consequently indirect cancer susceptibilities can be indicated by the increased appearance of fluorescent/luminescent cells (in the case of conservative homologous recombination between [Marker1] and [Marker2]) and/or the increased disappearance of cells with a different fluorescence/luminescence (in the case of non-conservative homologous recombination or replication slippage between [Marker1] and [Marker2] and consequently loss of [Ins2]).

The invention consequently relates to a process for analysing genotoxicities on the basis of the detection of conservative homologous recombination processes, non-conservative homologous recombination processes, of inactivating and reverse mutations, separately or in the same batch, wherein the above-mentioned cells which have been transfected with a vector according to the invention or the above-mentioned mammals, are brought into contact with a test compound, transfected with an I-SceI expressions plasmid, I-SceI being expressed by induction (e.g. by administering estradiol or tetracyclin), or are left untreated. Subsequently, DNA exchange frequencies can be determined by FACS analysis, fluorescence measurement, fluorescence microscopy, laser scanning cytometry, or by means of a luminescence detection reaction, with fluorescent or luminescent cells indicating these events as a function of the activity of the wild-type gene product of [Marker1] and [Marker2]. The loss of fluorescence or luminescence in individual cells of the population indicates non-conservative homologous DNA exchange events, replication slippage, or inactivating mutations, if [Ins2] of the vector used is the sequence encoding wild-type AFP or LE or it indicates inactivating mutations, if [Ins1] is an AFP/LE gene. Insofar as [Ins1] and [Ins2] are AFP or LE genes inactivated by mutation, the reverse mutations in [Ins2] or in [Ins1] are detectable by the appearance of a fluorescence or a chemiluminescence signal of the product of the wild-type gene concerned.

Moreover, the above-named vectors can also be used to characterise the importance of a gene with respect to its possible functions in maintaining or reducing the genetic stability in general, during DNA recombination or the formation of mutations in particular, with the above-mentioned process being used on cells or mammals with different status (status in this context meaning changes in the gene which affect the phenotype, i.e. in the simplest case a mutation which inactivates the gene product) with respect to the gene of interest (by mutation, deletion, over-expression etc.) to determine genotoxicities both in the presence of absence of a test compound. Finally, genetic rearrangements are detected by the appearance of fluorescence signals. Regarding the role of individual genes, the activity loss e.g. of the recombination regulator p53, leads to a decrease in the spontaneous homologous recombination events or those induced by a genotoxic treatment (e.g. the formation of a double strand break by I-SceI) (Xia, F. et al. (1994) Mol. Cell. Biol. 14: 5850-5857; Bertrand, P. et al. (1997) Oncogene 14: 1117-1122; Mekeel, K. L. et al. (1997) Oncogene 14: 1847-1857; Dudenhöffer, C. (1998) Mol. Cell.

Biol. 19: 2155-2168; 18: 5773-5784; Dudenhöffer, C. et al. (1999) Oncogene 18: 5773-5784; Saintigny, Y. et al. (1999) Oncogene 18: 3553-3565; Willers, H. et al. (2000) Oncogene 19: 632-639). When comparing isogenic cells, with or without p53, by use of the process presented in this invention on the basis of functional AFP detection, a lower number of fluorescent cells among the total cell population with p53 can be observed, corresponding to the recombination suppression by p53. In general, it is possible to determine, by exploiting existing cells or animal organisms with a different status regarding the gene of interest, the role of the gene product with respect to the genomic stability, genotoxicity, or cancer susceptibility by introducing into these cells or into these animals a vector for the determination of genotoxicities by physical or chemical transfection methods or by retroviral infection and by analysing the cells subsequently by FACS analysis, fluorescence microscopy, laser scanning cytometry, fluorescence measurement, or chemiluminescence detection reaction, wherein cells with a fluorescence or chemiluminescence corresponding to the activity of the wild-type gene product of [Marker1] and [Marker2] indicate DNA exchange events. The loss of a fluorescence or chemiluminescence activity in individual cells of the population moreover indicates DNA exchange events or inactivating mutations, if [Ins1] or [Ins2] of the vector used is the sequence encoding an alternative AFP or LE. Insofar as [Ins1] and [Ins2] represent AFP or LE genes inactivated by mutation, reverse mutations in [Ins2] or [Ins1] become detectable by the appearance of a fluorescence or luminescence signal of the product of the wild-type gene concerned. The status of the gene of interest can also be manipulated during the execution of the process according to the invention by using the coding sequence of the factor to be investigated or a mutant form thereof as [Ins1] or [Ins2] and, consequently, by introducing it together with the Marker genes into the cells or animals. When subsequently comparing cells which had been transfected with a vector with or without the corresponding [Ins1] or [Ins2] or infected retrovirally, the proportion of fluorescent or luminescent cells indicates the frequency with which DNA exchange events or mutations have taken place and thus allows conclusions to be drawn on the role of the gene product of interest in processes of genomic stabilisation or destabilisation, in genotoxic effects, and in the development of susceptibilities to cancer. Finally, the gene of interest can be “switched on or off” within one and the same cell and its role can be analysed within the same cell, as described for cells with different gene status. This is the case if [Prom2] belongs to the strictly inducible promoters such as those responsive to tetracyclin or estradiol. In addition, a transcription factor response to tetracyclin or estradiol must be expressed for these two examples and tetracyclin and/or estradiol must be added to the cells.

Moreover, a process is provided for the determination of the genetic stability or instability of a cell type or the recombination or mutation frequencies of a cell type, tissue type, or a eukaryotic organism or a process for characterising the genetic stability or instability of or the recombination or mutation frequency in cells, tissues, or mammals as a function of the physiological state thereof (e.g. cell cycle stage). In this process, the above-mentioned process for the determination of genotoxicities is used in which cells of the cell type or tissue type in question or, in general, of an eukaryotic organism are used after transfection or retroviral transduction with the above-mentioned vectors. Subsequently, DNA exchange frequencies can be determined by means of FACS analysis, fluorescence measurement, fluorescence microscopy, laser scanning cytometry, or by means of a luminescence detection reaction, with fluorescent or chemoluminescent cells corresponding to the AFP/LE activity of the wild-type gene product of [Marker1] and [Marker2] indicating these results. Loss of fluorescence or luminescence in the cells of the population indicates DNA exchange events, replication slippage, or inactivating mutations, if [Ins1] or [Ins2] of the vector used is the coding sequence of an AFP or LE. If [Ins1] and [Ins2] represent AFP or LE genes inactivated by mutation, reverse mutations in [Ins2] or in [Ins1] are detectable by the appearance of a fluorescence or luminescence signal of the product of the wild-type gene concerned. If a cell type, tissue type or the organisms of interest exhibits significantly higher DNA exchange or mutation rates in comparison with the average values of the controls (comparison with control cells, control tissues, or control organisms not containing these vectors), it will be classified as being genetically unstable. For tissue and organism analysis, there exists the possibility of obtaining cells from the living animal, e.g. from the blood, or the possibility of analysing certain tissues in the dead animal, e.g. by laser scanning cytometry.

Finally, the invention also relates to a process for the determination of the predisposition of a eukaryotic individual regarding the appearance and/or progression of cancer which, like breast cancer, is associated with genetic instabilities (Parshad, R. et al. (1996) Brit. J. Cancer 74: 1-5). In this process, the above-mentioned process for the determination of genotoxicities is carried out during which cells of the individual (e.g. from the blood, biopsy) are used which are transfected with the above-mentioned vectors or retrovirally transduced. The cells obtained from the subject are analysed after transfection by means of FACS analysis, fluorescence microscopy, laser scanning cytometry, fluorescence measurement or luminescence detection reaction, the cells with a fluorescence or luminescence corresponding to the activity of the wild-type gene product of [Marker1] and [Marker2] indicating DNA exchange events. The loss of fluorescence or luminescence in cells of the population indicates non-conservative DNA exchange events, replication slippage or inactivating mutations, if [Ins1] or [Ins2] of the vector used is the sequence encoding an AFP or LE. Insofar as [Ins1] and [Ins2] represent AFP or LE genes inactivated by mutation, reverse mutations in [Ins2] or in [Ins1] are detectable as a result of the appearance of a fluorescence or luminescence signal of the product of the wild-type gene concerned. Increased DNA exchange and mutation rates compared with the average values of cells of control persons are indicative of a predisposition for the appearance, the eruption or the progression of breast cancer and possibly also of other forms of cancer.

Finally, the invention relates to kits for carrying out the above-mentioned processes. A kit, e.g. for carrying out the process for the determination of genotoxicities, preferably contains cells, in particular mammalian cells which have been transfected with a vector, according to the invention, for the determination of genotoxicities, as well as suitable dilutions of a vector or an inductor (e.g. estradiol or tetracyclin) for I-SceI expression as a positive control. Instead of the transfected cells, the kit may also contain the vectors according to the invention or retroviral particles as such. Alternative kits for experimental performance contain suitable dilutions of a vector, according to the invention, for the determination of genotoxicities or a selection of vectors, according to the invention, a vector or inductor (e.g. estradiol or tetracyclin) for I-SceI expression, suitable transfection reagents and, optionally, reagents for carrying out the chemiluminescence reaction.

The invention presented here relates to a novel test system for the determination of genotoxicities on the basis of recombination markers on transfectable plasmids or with localisation on the chromosomes of mammalian cells. In this test system, the damage to the DNA is detected by a frequent appearance of genetic alterations directly in the damaged mammalian cells. The particular selection and arrangement of the individual sequence elements make it possible to detect all DNA recombination types known at present and, additionally, inactivating point mutations. In this way, a majority of all mutations is made detectable according to the invention and the sensitivity of the detection of genotoxicities is, thus, in turn maximised. The reconstitution of a gene coding for an intensively fluorescing protein (Clontech, Palo Alto, Calif., USA), e.g. the green fluorescent EGFP, and additionally or alternatively the retention or loss of another gene coding for a differently coloured fluorescent protein, e.g. the red DsRed, serves as the signal. The quantitative analysis can be performed by directly measuring the fluorescence in the damaged cells under the fluorescence microscope, in the FACS scan flow cytometer, or the laser scanning cytometer. Moreover, this detection method, based on the reconstruction and/or the loss of the autofluorescence of proteins, is designed by the choice and order of the sequence elements in such a way that it can be transferred into different mammalian cells and to living animals in order to be able to monitor DNA rearrangements in situ. The DNA rearrangements to be analysed relate to transgenic sequences which can be integrated in an intact manner into one of the cellular chromosomes by retroviral transduction. According to the invention, the Marker genes can be inserted in a linear manner and as individual copies as a result of this retroviral vector design and the individual chromatin context can be taken into account during measurements with cells and the animal model. In transient assays, too, i.e. after the transient introduction of the recombination construct by transfection, DNA rearrangements can be assessed with this test system in response to genotoxic treatment. For this reason, there is no need to establish stable individual cell clones for every measurement, in other words, primary cells, e.g. from biopsy materials, can be analysed. The incorporation of a recognition sequence for the meganuclease I-SceI from Saccharomyces cerevisiae into one of the Marker genes furthermore opens up the possibility of purposefully raising the basal rate of the DNA rearrangements by way of introducing the meganuclease into suitable test cells and by the resulting targeted generation of a double strand break (DSB) in one of the Marker genes (Rouet, P. et al. (1994) Proc. Natl. Acad. Sci. USA 91: 6064-6068). It is thus possible, according to the invention, to detect possible synergistic effects between the generation of DSBs/their repair and the noxae to be analysed with respect to mutation frequencies.

According to the invention, the test system is superior to the above-mentioned systems with respect to all the problematic aspects discussed. It has been established to detect genotoxicities in mammalian cells and is transferable to living animals as a transgene. In other words, the biotransformation of procarcinogens in the test system corresponds to that of the desired mammalian system. Moreover, no second organism is required for the detection. For the genotoxicity detection it is neither necessary to cultivate cells under selection pressure for several days nor to lyse or extract cells nor to apply another substance apart from the potentially genotoxic agent to be tested. Mutagenicity which, in this case, is indicated by the fluorescence signal, can be measured within hours after genotoxic treatment. In contrast to the cytogenetic methods (SCE detection, micronucleus assay), the Comet assay and the detection of increased DNA repair synthesis activities, the detection of fluorescent cells in a population of non-fluorescent cells is very simple and suitable for the analysis under high throughput conditions. The design of the test system does not impose any restrictions on the genotoxicity detection regarding certain stages of development or tissue types in the animal model.

In a study recently published by Jasin and colleagues (Pierce, A. J. et al. (1999) Genes & Development 13: 2633-2638), the analysis of gene conversion frequencies was measured as a function of the repair gene Xrcc3. The gene conversion test system used in this study was based on the reconstitution of a wild-type EGFP gene from two mutant forms of the EGFP gene. This principle is also an element of the invention described here. In contrast to the recombination test system of Jasin and colleagues, however, it is possible by means of the test system, according to the invention, to detect not only gene conversion and double crossover, i.e. conservative, non-deleting recombination events (see below) but also non-conservative single crossover, single strand annealing (SSA), and replication slippage as well as point mutations. In another embodiment of the test system, end joining can, moreover, be detected which links DNA ends via homologies of only a few base pairs. This had become possible by a combination of genes coding for two differently autofluorescing proteins (AFPs), by the design of the mutations and by the special arrangement of the individual DNA elements in the construct as a whole. An important progress was achieved in the system, according to the invention, particularly by refraining from expressing the AFPs as fusion proteins. In this way, it was possible to avoid the fluorescence intensity losses of the AFPs connected therewith. At the same time, the mutant gene segments which, after successful homologous DNA exchange, code for an AFP, e.g. EGFP, were maximised regarding their homologies by mutating only 4 base pairs encoding the amino acids in the chromophore region and/or the start codon into a stop codon only. Since no truncated genes were thus used here as the mutant genes, the homologous segments did not need to be extended via foreign sequences coding for an EGFP fusion, in order to guarantee sufficient exchange rates for the detection. In combination with the maximisation of AFP gene transcription by the extremely strong CMV promoter, the sensitivity of the system, according to the invention, is so high as a result of the measures listed that the limit of detection is below the rate of spontaneous DNA exchange rates. This has the effect that it is not only possible to measure genetic exchange processes following initiation by a targeted cleavage in one of the two gene variants by the meganuclease I-SceI, which is 100-10000 times more frequent (Liang, F. et al. (1996) Proc. Natl. Acad. Sci. USA 93: 8929-8933). In contrast to the gene conversion test system of Jasin and colleagues, it is possible, thanks to the present invention, to detect the results of different recombination types and potentially also of point mutations by the detection of differently coloured fluorescing cells in the FACS scan flow cytometer or the fluorescence microscope. Another study published recently (Slebos, R. J. C. and Taylor J. A. (2001) Biochem. Biophys. Res. Commun. 281: 2121-2129) describes a recombination test system which allows the reconstitution of a GFP gene by means of an EBFP gene. The advantage of the genotoxicity assay system, according to the invention, as compared to this recombination test system is, at the technical level, the complete omission of less strongly fluorescing fusion proteins, the exploitation of the 35 times stronger fluorescence intensity of EGFP in comparison with GFP (Clontech, Palo Alto, Calif., USA), and the maximisation of the homologies to 533 bp. The system, according to the invention, even offers the possibility of any desired extension of the homologous segments and consequently an increase in the DNA exchange events as a result of genotoxic treatment, since homologous sequences of any length can be inserted into the segments [Marker1] and [Marker2], if these flank a coding sequence for an autofluorescent protein or a bioluminescent enzyme, or an enzyme that converts chemoluminescence substrates in segment [Ins2]. Moreover, the test system, according to the invention, includes any type of gene inactivating DNA exchange and mutation event, by introducing a sequence encoding an autofluorescent protein or a bioluminescent enzyme, or an enzyme that converts chemoluminescent substrates into segment [Ins2]. Moreover, the genotoxicity detection system, according to the invention, provides the possibility of introducing a targeted DSB by means of I-SceI meganuclease and consequently of analysing genotoxic effects in association with or in comparison with DSB repair events. In addition, DNA substrates of the system, according to the invention, have been stably introduced into the chromosomes of mammalian cells as a result of which it can be used for the sensitive detection even of rare genetic alterations. This means that the process, according to the invention, is suitable for the sensitive and routine detection of repair responses to different genotoxic treatments. Proof of this possibility has been provided by experiments, whereas Pierce and colleagues as well as Slebos and colleagues carried out recombination measurements exclusively and analysed them again exclusively for events occurring at a frequency higher by several orders of magnitude following the introduction of a DSB by I-SceI meganuclease or after the introduction of 10⁵ to 10⁶ DNA copies by transient transfection. A significant improvement with regard to the commercial and consequently the automated utilisation of a corresponding genotoxicity determination system is provided in the system, according to the invention, also by the choice of mammalian cells which grow in a suspension culture and which, moreover, are resistant to active cell death yet are nevertheless p53 negative and consequently exhibit a recombination rate which is higher by several orders of magnitude (Mahdi, T., D. et al. (1998) Biol. Chem. 90: 15-27). Finally, the test system for the determination of genotoxicities, according to the invention, can be transduced via retroviruses. This allows not only the efficient transfer into new target cells but also the linear and single-copy insertion into the cellular genome (Coffin, J. M. (1996) in Fundamental Virology, Fields, B. N., Knipe, D. M. and Howley, P. M. (eds.), Lippincott-Raven Publishers, Philadelphia, 763-844). Since retroviruses are used for therapeutic gene transfer, it is, moreover, significant that this test system can also be used to analyse the stabilities or instabilities at the insertion sites of genomically integrated proviruses (position effects).

3.1 Importance of the Detection of DNA Recombination on Cellular Chromosomes

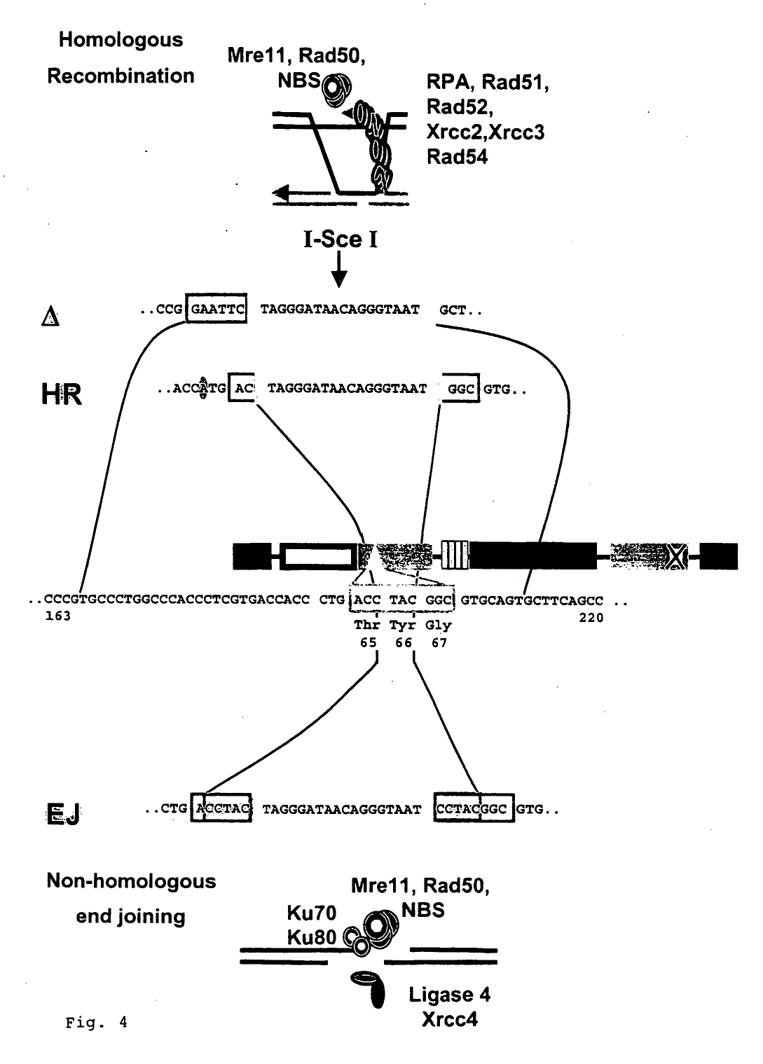

Recombination processes represent the final and irreplaceable repair mechanism, when DNA cross-links, DNA double strand breaks, or DNA gaps do not allow the missing information to be transferred from the complementary strand. In addition, individual strand breaks or as yet unrepaired lesions in the replication fork, i.e. during DNA synthesis in proliferating cells, also need to be removed by recombination (Haber, J. (1999) Trends Biochem. Sci. 24: 271-275; Cox, M. M. et al. (2000) Nature 404: 37-40). For this reason, recombinative repair processes represent one of the most important forms of DNA repair which, above all, respond to a wide variety of DNA damage. This makes the detection of recombination processes as an indicator of DNA damage upon the action of very different genotoxic agents or irradiation interesting. Recombination processes involved in repair need to be subdivided into various types, on the basis of their mechanism (Kanaar, R. et al. (1998) Trends Cell Biol. 8: 483-489): In the case of homologous recombination, identical or very similar sequences are copied which are present on the same chromatid (e.g. in the case of repetitive sequences), on the sister chromatid (especially during DNA synthesis), or on the homologous chromosome (especially during meiosis). In the case of eukaryotes, uninterrupted homologies of 134 to 232 base pairs are necessary for successful exchange (Waldman, A. S. and Liskay, M. (1988) Mol. Cell. Biol. 8: 5350-5357). Conservative homologous recombination processes lead to a complete reconstitution of the damaged sequences. This includes reciprocal double crossover and unilateral gene conversion. In the case of non-conservative homologous recombination events (nkHR), the DNA segments between the homologous DNA exchange partners are lost, i.e. they are the cause of deletions. This includes single crossover, single strand annealing, and replication slippage. In mammals homologous recombination represents the DSB repair pathway which is utilised in approximately one half of the events (Liang, F. et al. (1998) Proc. Natl. Acad. Sci. USA 95: 5172-5177). In proliferating cells, sister chromatid sequences or repeating sequences on the same chromatid almost exclusively serve as recombination substrates. For the basic strand exchange processes, it is above all the proteins Rad51, Rad52, Rad54, Xrcc2, and Xrcc3 which are required (compare FIG. 4 and reference Lambert, S. et al. (1999) Mutat. Res. 433: 159-168). Moreover, DSBs are fused by end joining (EJ), i.e. by simple coupling of the DNA ends, i.e. a ligation, or by coupling via very short homologies consisting of only a few base pairs. In this case, the enzyme complexes Mre11-Rad50-NBS, Ku70-Ku80, and ligase4-Xrcc4 play an important part (FIG. 4). The most important contributions to DSB repair in mammals are provided by gene conversion, SSA, and EJ (Critchlow, S. E. and Jackson, S. P. (1998) Trends Biochem. Sci. 23: 394-398; Lambert, S. et al. (1999) Mutat. Res. 433: 159-168; Johnson, R. D. and Jasin, M. (2000) EMBO J. 19: 3398-3407). In addition, EJ and gene conversion events may be coupled with each other which may have to do with the participation of Mre11-Rad50-NBS both in EJ and in the initiation of homologous recombination (compare FIG. 4).

Due to the particular suitability of homology-directed recombinative repair processes as indicators of DNA damage caused by carcinogens (Schiestl, R. H. (1989) Nature 337: 285-288; Brennan, R. J. et al. (1994) Mutat. Res. 308: 159-167; Carls, N. and Schiestl, R. H. (1994) Mutat.

Res. 320: 293-303; Aubrecht, J. et al. (1995) Carcinogenesis 16: 2841-2846; Schiestl, R. H. et al. (1997) Proc. Natl. Acad. Sci. USA 94: 4576-4581), the development of a recombination test system in mammalian cells represented the starting point for the invention described here. It was also an aim to make it possible, by the corresponding vector design, to insert the recombination markers of the test system into the cellular genome in a linear manner and as a single copy by retroviral transduction, in order to be able to make a clear statement on the type of recombination (e.g. EJ versus homologous recombination) (compare section 3.5).

3.2 Targeted Initiation of Recombination Events

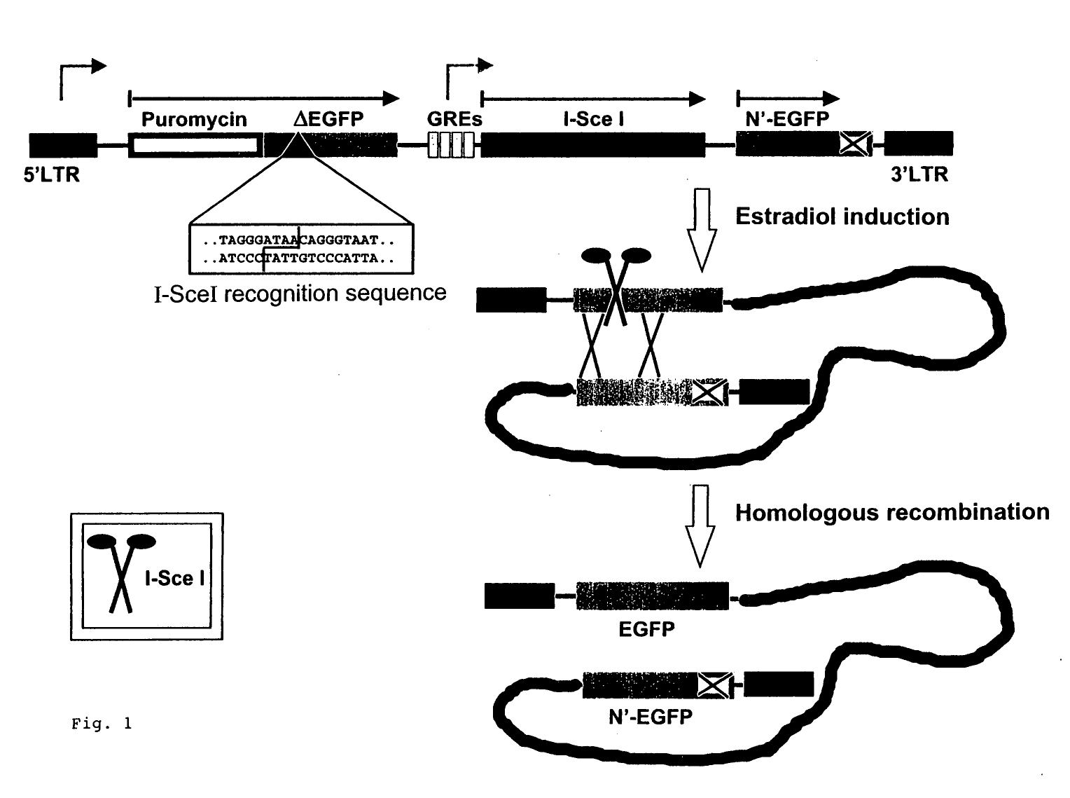

Another important aspect of the assay design was that it had to be possible to initiate recombination events not only by radiation and genotoxic agents but also by the specifically targeted introduction of a DSB into one of the marker genes. This makes it possible, on the one hand, to establish the test, serves as an internal standard and, moreover, allows to detect possible synergies on DNA repair events between the DSB introduced in a targeted manner and the effects of genotoxic treatments. To initiate DNA exchange events by targeted DSBs, I-SceI meganuclease was chosen (Liang, F. (1996) Proc. Natl. Acad. Sci. USA 93: 8929-8933). I-SceI is an endonuclease from S. cerevisiae with a recognition sequence 18 base pairs in length. According to statistical calculations, the recognition sequence is unlikely to be found naturally in mammalian genomes. The cDNA was tailor-made, by Dr. Maria Jasin, Sloan-Kettering Institute, New York, for the expression in higher eukaryotes and N-terminally fused with the hemagglutinin peptide epitope (HA) for the monoclonal antibody 12CA5 (Roche). Dr. M. Jasin demonstrated that the generation of DSBs by I-SceI in hamster cells stimulates homologous recombination events 10²-10⁴ fold. In order to be able to produce DSBs locally in the desired recombination marker, in the EGFP gene in the simplest case, the I-SceI recognition sequence was firstly inserted by molecular biology into one of the EGFP marker genes (compare section 3.3) and the meganuclease was secondly expressed (compare FIG. 1).

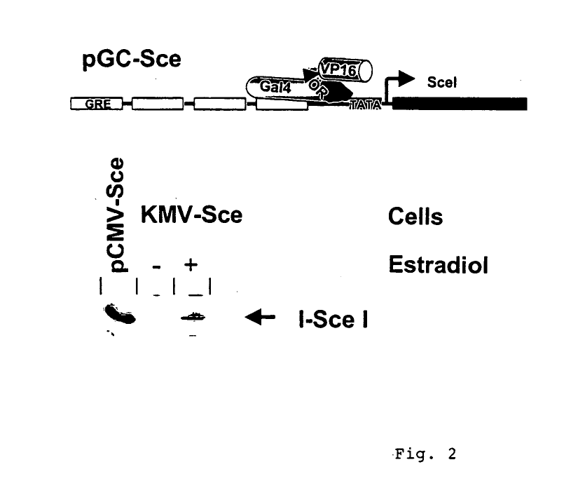

In order to achieve controlled meganuclease expression because of the antiproliferative effect of DSBs, the extremely reliable, β-estradiol-inducible expression system was selected according to a preferred embodiment of the invention (Braselmann, S. et al. (1993) Proc. Natl. Acad. Sci. USA 90: 1657-1661; Dudenhöffer, C. (1999) Oncogene 18: 5773-5784). The expression system couples the so-called Gal4ER transcription factor to an expression cassette controlled by a Gal4ER-responsive promoter (compare FIG. 2). Gal4ER represents a fusion protein of the DNA binding domain of the Gal4 transcription factor with the strong, viral VP16 transactivator domain and a β-estradiol binding domain of the human β-estradiol receptor, making Gal4ER functionally inducible as transcription factor by β-estradiol. This principle of conditional expression permits an inducible increase in protein expression by at least one order of magnitude. As part of the present invention, MMV, WMV, and KMV lines derived from the parental methyl cholanthrene-induced Balb/c-mouse fibrosarcoma cells (DeLeo, A. B. et al. (1977) J. Exp. Med. 146: 720-734), human WTK1 lymphoblasts (Xia, F. et al. (1994) Mol. Cell. Biol. 14: 5850-5857), and from human myelogenic K562 leukemia cells (Andersson, L. C. et al. (1979) Int. J. Cancer 23: 143-147) were established for testings, in which the transcription factor Gal4ER was expressed constitutively, i.e. permanently. This was achieved via electroporation of the cells with the expression plasmid pMVGalERVP and subsequent isolation of individual cell clones in soft agar by means of the pMVGalERVP-encoded antibiotic resistance to neomycin. Subsequently, KMV-derived cell lines were established which expressed I-SceI after β-estradiol induction. For this purpose, KMV cells were electroporated with the vector pGC-Sce (FIG. 2) and a plasmid providing resistance to hygromycin, and individual cell clones were isolated under antibiotic selection pressure. Inducible I-SceI expression was detected in Western blot experiments. As regards the stability and controllability of the Gal4ER expression, no changes were observed over a period of several months. As demonstrated in FIG. 2, in GalER expressing cells it was possible in this way to switch on and off the expression of the I-SceI meganuclease within 4 hours, i.e. by the simple addition of β-estradiol (≧50 nM) to the medium. For this reason, as in pGC-Sce, the sequences coding for I-SceI meganuclease were positioned downstream of the recognition sequences of the Gal4 transcription factor (GREs) within the retroviral vectors with the AFP recombination markers, according to the invention (compare section 3.3). β-estradiol-dependent expression was demonstrated via an easily detectable marker protein, namely p53, again in the context of the recombination vector. Overall, the prerequisites were thus fulfilled, to initiate recombination in cells by I-SceI meganuclease following the administration of β-estradiol at a predetermined time, e.g. in the cell cycle. Alternatively, the I-SceI enzyme can be produced transiently in mammalian cells by transfection with an expression plasmid (compare section 5 example 4). It could be shown that, following transfection, 80% of the molecules with an I-SceI recognition sequence are cleaved (Rouet, P. and Jasin, M. (1994) Mol. Cell. Biol. 14: 8096-8106).

3.3 Measuring Recombination by the Reconstitution of an AFP Gene

In principle, a test system for homologous recombination must possess two homologous DNA sequence segments as recombination marker. In one embodiment, the two sequences represent mutant variants of a gene such that the wild-type gene and thus the encoding wild-type protein can be reconstituted only by homologous DNA exchange. In the very frequent case of gene conversion, i.e. unilateral gene exchange, the sequence segment, which supplies the information for reconstitution, is referred to as the donor and the second gene as the acceptor. In the case of the introduction of a specifically targeted DSB, e.g. via I-SceI, into one of the two genes, this becomes the acceptor.

In the first stage, the strong green autofluorescence of the EGFP protein was used to detect homologous recombination events (compare FIG. 1). The detection system, according to the invention, is equally suitable for using other similarly intensively fluorescing AFPs from Clontech, Palo Alto, Calif., USA, such as EBFP (blue), ECFP (cyan), EYFP (yellow), DsRed (red), or the AFPs from other manufacturers. The maximum absorption of light by EGFP is at 488 nm and it emits green light at 507 nm. The fluorescence of the protein is extremely stable and consequently highly suitable for use as a reporter for the detection of individual fluorescent cells in a population of non-fluorescent cells by fluorescence-activated cell sorting (FACS) or by fluorescence microscopy. In order to be able to quantify recombination frequencies by detecting individual fluorescent cells in the FACS scan flow cytometer, sufficient sensitivity must be guaranteed. For this purpose, preliminary mixing experiments were carried out with non-fluorescent mKSA tumour cells and with murine mKSA tumour cells which produce EGFP after the stable integration of the EGFP expression cassette of the Clontech vector pEGFP-N1 into the cellular genome. The evaluation in the Calibur FACS scan flow cytometer from Becton Dickinson showed that, for EGFP expressed by means of the CMV promoter, the fluorescence intensity is more than two orders of magnitude higher than the slightly orange endogenous fluorescence of the cells. Furthermore, by means of EGFP, it was possible to detect fluorescent cell sub-populations in a dilution of up to at least 10⁻⁵.

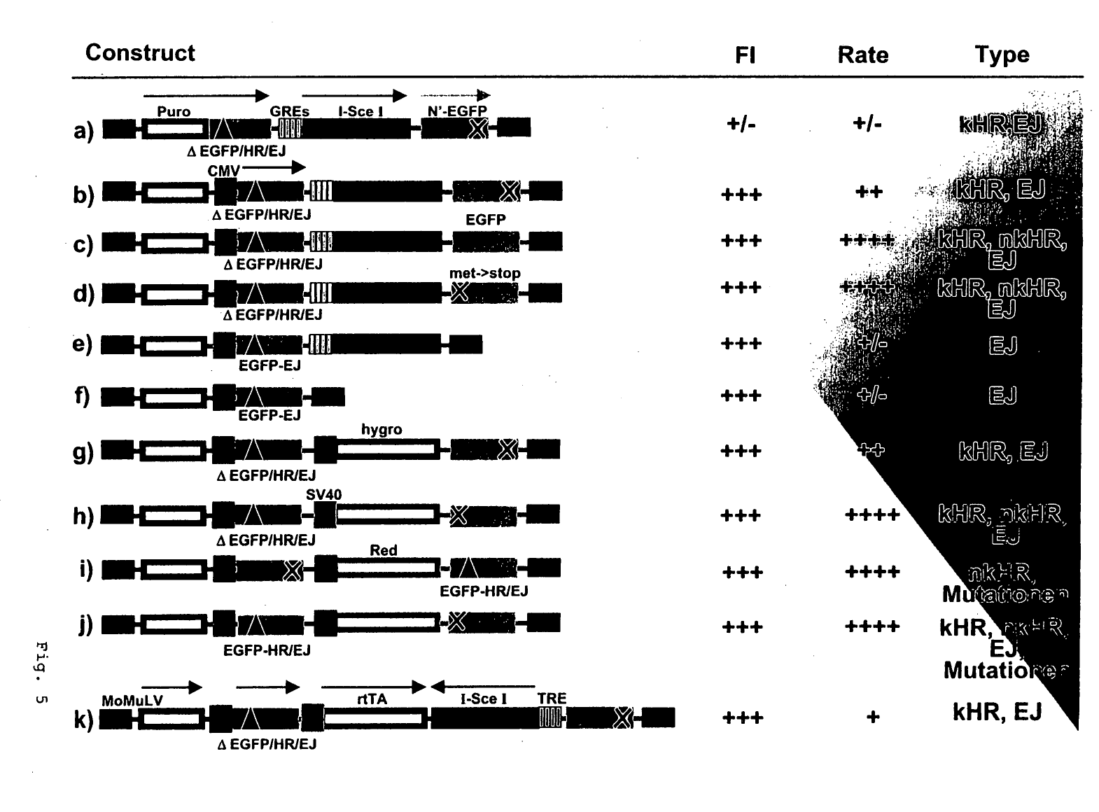

For the reconstitution of a wild-type EGFP gene, mutant variants of the EGFP gene were used as recombination markers. The first variant, _{Δ}EGFP, which represents an acceptor substrate, was produced by PCR technology from a cDNA fragment 731 base pairs in length with the coding region of EGFP (starting 2 base pairs upstream of the start codon and terminating 9 base pairs downstream of the stop codon) by replacing the base pairs coding for the chromophore amino acids 65-67, 29 additional base pairs 5′ and 7 base pairs 3′ to it by the recognition sequence of meganuclease I-SceI and the restriction endonuclease EcoRI (compare FIG. 4). The second variant, N′-EGFP, a donor substrate, represents a fragment shortened by 278 base pairs at the 3′ end within the region coding for EGFP, so that the resulting protein lacks chromophore coating for protection against quenching effects. Thus, N′-EGFP covers the region mutated in _{Δ}EGFP and can be used as a matrix for homologous DSB repair. _{Δ}EGFP was inserted into the reading frame of the puromycin resistance gene, such that the ΔEGFP protein is translated in fusion with puromycin-N-acetyl transferase following mRNA synthesis, starting out from the viral 5′-LTR promoter (compare section 3.5). N′-EGFP was placed in series downstream of _{Δ}EGFP such that double crossover and gene conversion events exclusively can reconstitute an intact wild-type EGFP gene. A spacer had to be introduced between the recombination markers in order to guarantee the DNA flexibility necessary for DNA exchange. In the first version of the invention, the complete I-SceI expression cassette was inserted at this position (compare FIG. 1 and FIG. 5a and section 3.2). Integration of the puromycin resistance gene PAC prevents inhibition of protein biosynthesis in the presence of the antibiotic puromycin. This allows the establishment of independent cell clones which carry the construct for the detection of recombination events illustrated in FIG. 1 and FIG. 5a stably integrated into the cellular genome.

Another critical advantage of the test system for DNA exchange processes based on EGFP, according to the invention, is the short reaction time since, in this way, genotoxic effects can be detected without being distorted by long evaluation phases or by selection processes. The period between the termination of the recombination process and the detection of fluorescent cells was assessed as follows: Gal4ER expressing KMV cells were electroporated with the reporter vector pGC-EGFP prepared for this purpose, estradiol was added to the cells after a one day recovery period, and, subsequently, the development of autofluorescence was checked at various times. After 4 h only, fluorescent cells were clearly distinguishable from non-fluorescent cells. This represents significant progress compared with conventional cell cloning strategies which require reaction periods ranging from several days to several weeks.

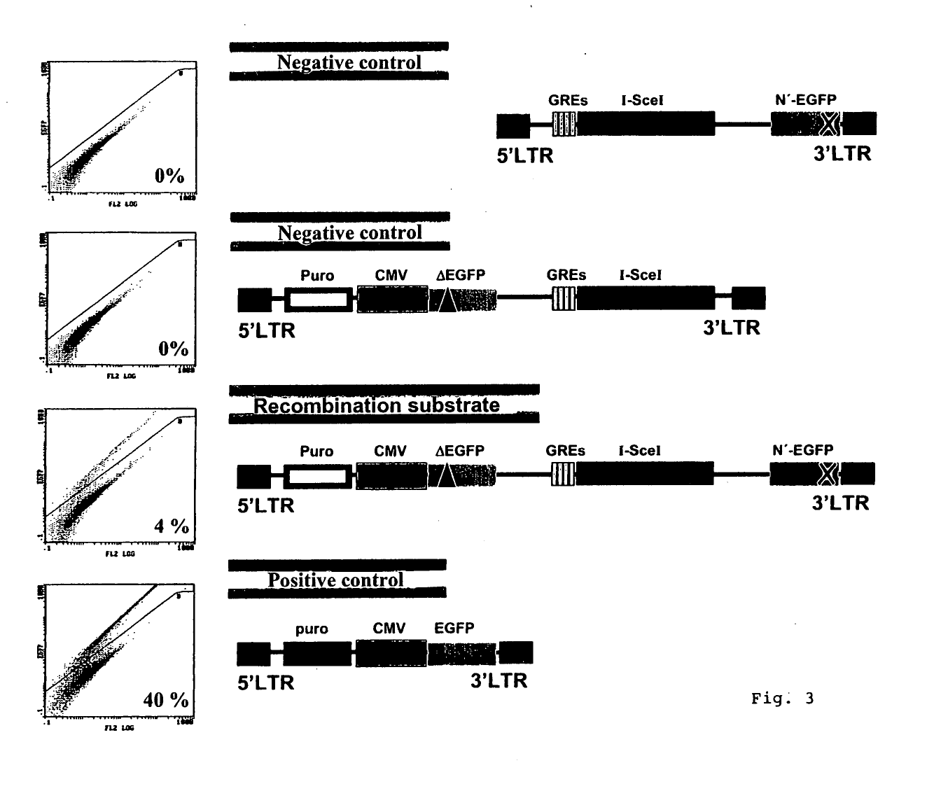

According to the invention, recombination experiments were initially carried out in transient electroporation assays in KMV target cells using the recombination construct from FIG. 1. After activation of the I-SceI meganuclease, a slight shift in the fluorescence intensities was observed in the FACS scan cytometer for a few percent of the cells. Although this shift in the histogram, which is dependent on I-SceI and the recombination substrate, indicated reconstitution of the wild-type EGFP gene achieved by recombination, quantification was difficult as a result of this weak fluorescence increase. To be able to make clear statements about successful EGFP gene reconstitution, an effort was made to achieve an increase in the fluorescence intensity by 1-2 orders of magnitude. A clearer separation of the endogenous orange autofluorescence from the green fluorescence in the dot plot versus the histogram (FL2 versus EGFP in FIG. 3) and visualisation under the fluorescence microscope was to be made possible. For this reason, strategies were devised for maximising the fluorescence intensities and the recombination frequencies.

3.4 Maximising the Signals for Detecting Homologous Recombination

Since EGFP is at present the autofluorescent protein with the strongest light emission, it was not the AFP type itself which was modified but, instead, its type of expression. Interestingly enough, when the fluorescence intensity of the puromycin-N-acetyl transferase EGFP fusion protein was directly compared with EGFP, it was found that the fusion protein exhibited a fluorescence which was 10 times less intense. This observation applies in different degrees to all EGFP fusion proteins, as has been increasingly realised by those skilled in the art. In addition, the comparison of the promoter strength of the retroviral MPSV-5′LTR and the CMV promoter from cytomegalovirus uncovered an expression rate that was 3-4 times higher for the CMV promoter. Consequently, the recombination acceptor _{Δ}EGFP and the puromycin resistance gene were separated and the recombination construct supplemented by a CMV promoter between the puromycin resistance gene and the _{Δ}EGFP gene (compare FIG. 3 and FIG. 5b). For the constructs thus modified, it was possible to clearly identify green fluorescing cells in the dot plot in the upper left hand triangle after FACS analysis, as shown in FIG. 3. This fluorescent sub-population contained cells after successful recombination which was caused by the transient introduction of the plasmids into GalER-VP expressing KMV cells by electroporation and by estradiol treatment, i.e. I-SceI activation. At last, the fluorescence intensity now was also sufficient for detecting green fluorescent cells under the immunofluorescence microscope. In the analysis of negative control constructs which lacked one of the two EGFP gene variants, no fluorescent cells could be detected. This made it clear that intact EGFP gene sequences had been reconstituted by recombination between the targeted acceptor substrate and the donor substrate. The recombination frequency was 4%, based on the cell population as a whole, or 10%, based on the transfected cells. Electroporation with a construct containing a wild-type EGFP gene indicated electroporation efficiencies of 40% of the cells (FIG. 3).

Apart from increasing the EGFP fluorescence intensity, the rates of homologous recombination were increased by maximising the homologous regions between the donor and the acceptor substrate (Waldman, A. S. and Liskay, M. (1988) Mol. Cell. Biol. 8: 5350-5357). For this purpose the regions in the acceptor substrate homologous to the donor substrate were enlarged by replacing only 4 base pairs within the region coding for the chromophore amino acids by the I-SceI recognition sequence (EGFP-HR in FIG. 4) instead of a total of 45 base pairs as in the case of the acceptor _{Δ}EGFP. Secondly, the entire wild-type EGFP-cDNA region, 720 base pairs in length, was used as donor substrate instead of the N′-EGFP, 443 base pairs in length, in order to enlarge the homology to the acceptor substrates _{Δ}EGFP and EGFP-HR within the 3′ portion (compare FIG. 5c). In this order, i.e. without a separate promoter, wild-type EGFP cannot be expressed polycistronically in eukaryotes. After introducing the corresponding constructs into KMV cells by electroporation and activating I-SceI meganuclease by estradiol, similar recombination frequencies were measured with EGFP-HR as for _{Δ}EGFP, whereas a substantial increase to 30% of the transfected cells was achieved with wild-type EGFP. However, a small number (1-2%) of green fluorescent cells was also observed in experiments with negative controls free of acceptor substrate. This means that it was possible to produce wild-type EGFP either by cryptic splicing or by internal initiation of protein translation. In order to exclude this possibility, one further improvement was integrated into the system, however, without changing the high recombination frequencies. For this purpose, the start codon of the wild-type EGFP gene in the acceptor gene portion was converted by PCR technology into two successive stop codons (FIG. 5d). In this way, maximal homologies were retained while, simultaneously, EGFP expression was completely suppressed except after successful recombination. The new donor EGFP gene was named as met->stop. 18-38% of the electrotransfected cells underwent DNA exchange processes with the corresponding construct. In this context it should be noted that, simultaneously with enlarging the homologies by the wild-type EGFP or by the met->stop gene in contrast to N′-EGFP, non-conservative homologous recombination was made possible in this order (compare section 3.5). The reason is that 189 base pairs 5′ to the I-SceI recognition sequence are homologous between the met->stop gene and the N′-EGFP gene and are available for non-conservative homologous recombination processes. Overall, the 7 fold increase in recombination frequencies with the wild-type EGFP or the met->stop gene, in comparison with the N′-EGFP gene as donor, could be explained both by the extension of the homologous pairing regions and by the inclusion of non-conservative recombination processes.

3.5 Potential for the Detection of Conservative and Non-Conservative Homologous Recombination Events, End Joining, and Point Mutations

Spontaneous gene conversion rates, as measured in immortalised mammalian cells, amount to 10⁻⁵ to 10⁻⁶ per cell. If the order of recombination substrates is such that, apart from conservative events such as gene conversion, non-conservative events such as SSA can take place, an increase in the spontaneous rates occurs so that these range between 10⁻⁴ and 10⁻⁵ (Lambert, S. et al. (1999) Mutat. Res. 433: 159-168; Moynahan, M. E. et al. (1999) Mol. Cell 4: 511-518; Dronkert, M. L. G. et al. (2000) Mol. Cell. Biol. 20: 3147-3156). The loss of the regulatory function of the tumour suppressor p53 during homologous recombination in mammalian cells, such as in K562, WTK1, or MethA cells used here, leads to an increase by at least one order of magnitude (Akyüz et al. (2000) Gen. Ther. Mol. Biol. 5, 1-17). This means that in the case of the detection of all types of homologous recombination, spontaneous recombination frequencies and undoubtedly those increased by noxae, can be detected directly in the FACS scan flow cytometer by using the assay system according to the invention.

In order to create the possibility of detecting DNA rearrangements completely independently of I-SceI meganuclease but e.g. as a function of environmental toxins, food toxins, or chemotherapeutic agents, the recombination construct was completely freed from the I-SceI expression cassette in further variants and replaced by a hygromycin resistance gene with SV40 or CMV promoter (FIGS. 5g and h). In the following experiments, it was now also impossible to measure spontaneous DNA exchange rates with this new variant. To be representative of strong DNA damage by exogenous factors, the I-SceI meganuclease was produced in parallel in a transient manner in the target cells by electroporation with an appropriate expression construct.

In order to create optimum conditions for non-conservative recombination events, in particular in view of the detection of exchange events independent of I-SceI, i.e. rarer exchange events, a new construct was produced which was tailor-made for measuring this most frequently occurring type of homologous recombination (Lambert, S. et al. (1999) Mutat. Res. 433: 159-168). At the same time, this construct was to be used to assess the contribution made by non-conservative recombination events to the exchange rates in the construct with the met->stop donor substrate. For this purpose, the EGFP-HR gene was placed, in contrast to the previous order of sequences, 3′ to the N′-EGFP gene and the two EGFP gene variants were separated by an expression cassette for the wild-type DsRed gene (FIG. 5i, section 5 example 1 and section 6 of the sequence protocol). In this way, a sequence of 242 base pairs with homologies to the N′-EGFP gene is available in the EGFP-HR gene between the I-SceI recognition sequence and the 3′ end. Single crossover, SSA, or replication slippage are capable of reconstituting an intact wild-type EGFP gene via these sequences. These non-conservative recombination events would simultaneously be accompanied by the loss of the DsRed gene and could consequently be detected both via green fluorescence and via the loss of red fluorescence. However, erroneous non-conservative recombination events which do not reconstitute an intact wild-type EGFP gene can thus be detected via the loss of red fluorescence. Point mutations in the DsRed gene which interfere with the autofluorescence of the gene product should thus also be detectable. For full autofluorescence to be deployed, AFPs require perfect folding of the protein, so that numerous small changes within the coding sequences accompany the loss of fluorescence (Cubitt, A. B. et al. (1995) Trends Biochem. Sci. 20: 448-455). An interaction between the sequences derived from EGFP and those derived from DsRed need not be expected since the EGFP gene was isolated from the jellyfish Aequorea Victoria while the DsRed gene originates from the sea anemone related Discosoma sp., i.e. no significant homologies exist between the two genes. The possibilities of promoter interferences occurring between the two CMV promoters upstream of the N′-EGFP gene and/or the DsRed gene could be excluded right from the beginning on the basis of similar experiments carried out by colleagues (Dr. Geoffrey Margison, Manchester, personal memo). This principle of analysis can be implemented in the case of a restriction to the highly frequent inactivating events by using any desired homologous sequences, i.e. those which do not necessarily code for AFPs, in a manner flanking the sequence segment [Ins2], in this example the DsRed gene.

To confirm the operating principle, an investigation was carried out, following the transient electroporation of K562 cells with the construct i of FIG. 5 with the EGFP-HR gene or a control construct without [Marker1], to determine the transfection efficiency via red fluorescence in the FACS scan flow cytometer following excitation at 488 nm for green EGFP fluorescence or for red DsRed-fluorescence. Among the tranfected cells, 4% of the cells exhibited green fluorescence. In the negative controls with control construct, no green-fluorescent cells were observed. This means that, in the transient electroporation test, the rates of wild-type EGFP gene reconstitution were similarly high for construct i as for construct h in FIG. 5, which, in contrast to i, indicates not only non-conservative homologous recombination but also gene conversion, i.e. the rates were 3-12%, again with respect to transfected cells. Following coelectroporation of the construct i from FIG. 5 or a derived construct with the wild-type EGFP gene in the [Marker1] sequence segment and an expression plasmid for meganuclease I-SceI, it was possible to detect increased rates for the wild-type EGFP gene reconstitution, namely of 13% in transfected cells, by introducing a DSB into the EGFP-HR gene. In comparison, the recombination frequency, induced by I-SceI, for the construct h in FIG. 5 was 6-22%, based on the transfected cells. Among the transfected cells, there was a decrease of the red-fluorescent cells by 56% in the absence of 1-SceI and by 76% in the presence of I-SceI. The latter amounted to 98% for the construct with the wild-type EGFP gene in the [Marker1] sequence segment. This means that the inactivating recombination and mutation events were up to 14 times higher in the absence of I-SceI than the values for the reconstituting events. As shown here by means of the wild-type EGFP genes in comparison with the N′-EGFP gene, another increase in the inactivating events can be achieved by extending the homologous regions. This represents another important increase in sensitivity for the genotoxicity determination. Further possibilities for improvement are provided by optimising the excitation wavelength (488 nm for EGFP versus 558 nm for red). Further, inactivating events can be recognised, following stable integration of the constructs into the cellular genome, by the complete loss of red fluorescence and not just by the decrease in the fluorescence signal below a threshold value. The laser scanning cytometer made by Compucyte appears to be a suitable tool in particular for this application, i.e. for detecting individual, non-fluorescent cells in a population of fluorescent cells since this device combines the advantages of individual cell analysis under the fluorescence microscope with the advantages of population analysis in the FACS scan flow cytometer. Another improvement in this test consists of the use of two genes coding for AFPs with excitation and emission spectra which are even more clearly separated from each other than EGFP and Red. Future non-homologous variants of yellow and cyan fluorescent proteins would be suitable candidates for this. In addition, the use of any desired, though homologous sequences, in the sequence segments [Marker1] and [Marker2], and consequently also sequences with sequence homologies of any desired length, provide an interesting possibility for improvement for the detection of inactivating events in the [Ins2] sequence segment.

As mentioned above, EJ also represents an important form of DSB repair in mammalian cells (Critchlow, S. E. and Jackson, S. P. (1998) Trends Biochem. Sci. 23: 394-398). In order to be able to measure EJ in association with the formation of DSBs by the meganuclease I-SceI and, in association with EJ, genotoxicities, a substrate designed specifically for the detection of this recombination type was generated. Since, in contrast to the homologous DNA exchange, two homologous partners are not required in the case of EJ, the recombination constructs illustrated in FIGS. 5e and f contain only one recombination marker gene, namely EGFP-EJ. To introduce a targeted DSB, EGFP-EJ again contains an I-SceI recognition sequence in the EGFP sequence which codes for chromophore amino acids (compare FIG. 4) and, moreover, an I-SceI expression cassette which can be activated by Gal4ER-VP in construct e of FIG. 5. In addition, 5 base pairs of the EGFP sequence were duplicated so that homologies 5 base pairs in length exist 5′ and 3′ to the I-SceI recognition sequence. In the case of EJ, these short homologies serve the purpose of reconstituting the wild-type EGP sequence after the introduction of a DSB by I-SceI. In this case, too, the operating principle was examined following transient electroporation of the construct in KMV cells. The production of the I-SceI meganuclease took place via the administration of estradiol or via coelectroporation using an I-SceI expression plasmid. The rates for successful EJ were again derived from the proportion of green fluorescent cells and were determined by means of the FACS scan flow cytometer. With 1-3% in transfected cells, they were lower than the values determined for homologous recombinations types. In order to integrate in general, in addition to homologous recombination, also the possibility of EJ and homologous DNA exchange coupled to EJ (Johnson, R. D. and Jasin, M. (2000) EMBO J. 19: 3398-3407), EGFP or EGFP-HR were replaced by EGFP-EJ in the existing recombination constructs, in additional variants (FIG. 5 a)b)c)d)g)h)i)k)).

Overall, as part of this invention constructs were generated which permit the detection of conservative homologous recombination processes, EJ, and non-conservative homologous recombination processes or of non-conservative homologous recombination processes and mutations in response to genotoxic treatment. Finally, the synthesis of the optimum variants in h and i, as illustrated in FIG. 5j, was to unify all the above-mentioned possibilities for detecting genetic changes as an indicator of genotoxicities. In addition, this optimised variant in FIG. 5j particularly also retains the order of the two EGFP gene variants illustrated in h and the maximal spontaneous recombination rates thus reached.

In comparison with the test systems for homologous recombination of other scientists (Pierce, A. J. et al. (1999) Genes & Development 13: 2633-2638; Slebos, R. J. C. and Taylor J. A. (2001) Biochem. Biophys. Res. Commun. 281: 2121-2129), the present invention of a genotoxicity detection system is technically superior in that an AFP fusion protein is avoided and intensively fluorescing AFPs are used exclusively and, moreover, homologous sequence regions can be maximised up to any desired length, thus achieving a very high sensitivity. Moreover, the advantages consist of the fact that the possibility exists of detecting different recombination types, mutations, and, above all, also the particularly frequent gene-inactivating events. Moreover, there is the possibility of the targeted introduction of DSBs by I-SceI into the genome of living cells without the need of transfection with an expression plasmid. Finally, there is the advantage of the transfer of the test constructs by means of retroviruses (compare section 3.6). Moreover, the cell type selected fulfills the prerequisites for automation by cultivation in suspension culture and by insensitivity to apoptosis with simultaneously high rates of DNA rearrangement.

3.6 Application Potential of the Test System for the Detection of Genotoxicities

The operating principle of the recombination and genotoxicity detection was confirmed also following the stable integration of the constructs into the cellular genome. In this way, murine MethA-, human WTK1, human K562, and GalER-VP producing KMV cells were analysed. In comparison with the transient assays following electroporation with the recombination constructs, lower recombination rates can be expected following the stable integration into the cellular genome since transfected DNA is naked, i.e. freely accessible to repair enzymes without chromatin packaging, but above all also since higher copies number of recombination substrates are present per cell following transfection. The recombination markers were inserted stably into the genome by electroporating the cells with the corresponding DNA, individual cell clones resistant to puromycin were isolated, and the integrity of the recombination construct was confirmed via PCR analyses of genomic DNA from the individual cell clones. For the construct with EGFP-EJ acceptor described in FIG. 5h, recombination events were detected via EGFP fluorescence in 6 independent clones in 0.2-0.6% of the cells electrotransfected with an I-SceI expression plasmid. The spontaneous recombination rates amounted to 10⁻⁵. The postulated DNA rearrangements were verified by genomic PCR for the corresponding cell populations following successful DNA exchange, in analogy to the detection of the integrity of the recombination constructs following integration into the cellular genome.

Etoposide, a chemotherapeutic agent and topoisomerase II inhibitor, can trigger DSBs but is not detected by the Ames assay. In order to provide evidence for the applicability of the test system according to the invention as genotoxicity detection system, the effect of ionising radiation and etoposide was investigated. For this purpose, 7 different clones derived from K562 cells, with test constructs stably integrated into the genome (h in FIG. 5), were irradiated with 500 rad and the fluorescence of 30,000 cells was analysed in the flow cytometer 24 h, 48 h, 72 h and 96 h after treatment in the flow cytometer in comparison with untreated cells. The results have shown that the appearance of green-fluorescent cells following irradiation was observed in two cell lines at more than two points in time, whereas no fluorescence signals were detected in untreated cells. Similarly, the appearance of green-fluorescent cells was recorded after a two hour treatment with etoposide (100 μM). The rates measured were 0.3-1×10⁻⁴. This means that the test system, according to the invention, creates the preconditions for assessing the effects of radiation, chemotherapeutic agents, or other noxae in human cells as an increase in the DNA exchange rates.

In order to permit the efficient and controlled transfer of the test constructs into any desired mammalian cell, these were, as a principle, generated within a retroviral vector structure. As a basis for the vectors illustrated in FIG. 5a to j, the retroviral vector p5NM derived from the genome of the myeloproliferative sarcoma virus MPSV, developed by Dr. Carol Stocking, Heinrich Pette Institut Hamburg, was used. The recombination markers were cloned in between the 5′LTR with the packaging signal and the 3′LTR, so that sequences positioned between the LTRs can be packaged in retroviruses in the corresponding helper cell lines and subsequently integrated into the genome of the desired cells via retroviral transduction. Compared with chemical and physical methods of gene transfer, this represents the major advantage that the linear order of the marker sequences is retained and multiple tandem insertions such as those frequently encountered, above all after calcium-phosphate coprecipitation, are avoided. Maintaining the order of the individual elements within the vectors in turn is the precondition for the DNA exchange processes to be detected. MPSV retroviruses, in comparison with the MoMuLV viruses, moreover, have the advantage that LTR-controlled expression is possible also in embryonal cells (Akgün, E. et al. (1991) J. Virol. 65: 382-388).

To produce infectious retrovirus particles, cells of the helper cell line PA317 were electroporated with the vectors in b, d, h, i, and k in FIG. 5, including negative controls devoid of recombination markers and positive controls containing wild-type EGFP. PA317 represents an amphotrophic retrovirus helper cell line, i.e. it carries all the genes for packaging replication-deficient retroviruses which are capable of infecting cells of mice, human beings, chickens, dogs and cats (Miller, A. D. and Buttimore, C. (1986) Mol. Cell. Biol. 6: 2895-2902). Since the gag, pro, pol, and env genes are missing in retroviral vectors, new virus particles are not formed in infected host cells. Single PA317 cell clones were isolated by means of puromycin selection and the linear insertion of the recombination markers into the genome of the individual PA317 lines was proven via PCR analyses. The infectiousness of the retroviruses in the supernatants of the cell line concerned was investigated and demonstrated for the types in b, d, and k in FIG. 5. Following the infection of murine MethA and/or K562 cells with these new retroviruses, PCR analyses carried out on the genomic DNAs proved the integrity of the sequences inserted between the LTRs into genome. In addition, MethA and K562 cells which had been infected with viruses of the wild-type EGFP-containing positive controls to vector types b to j in FIG. 5 exhibited clearly visible EGFP autofluorescence. Since proviruses are inserted as individual copies into the cellular genome, this means for future experiments for the determination of genotoxicities that, in the vector context presented here, the reconstitution of a single wild-type EGFP gene copy per cell is sufficient to indicate the effect of a genotoxic treatment.

Finally, in the context of the present invention, contructs were made on the basis of the retroviral vector pRetroOn from Clontech, which contain, within a single vector, elements for the tetracyclin-inducible expression of I-SceI meganuclease, for the constitutive expression of the tetracyclin-dependent transcription factor rtTA, and for the recombination substrates derived from EGFP (FIG. 5k). pRetroOn is derived from the murine Moloney leukemia virus (MoMuLV). pRetroOn permits tetracyclin-inducible I-SceI expression via a promoter (TRE) which is activated in the presence of tetracyclin or doxycycline by the transactivator reversely controlled by tetracyclin (rtTA). The sequences coding for rtTA are situated, in the case of these constructs, between the acceptor substrates ΔEGFP, EGFP-HR, or EGFP-EJ and the I-SceI gene. In other respects, the structure corresponds to the variant illustrated in b in FIG. 5. These and constructs derived therefrom have the advantage that, if desired, the I-SceI expression should be controllable also in the animal model since, in contrast to estradiol, it does not naturally occur in mammals.

The test system presented here in its different embodiments also creates good conditions for a possible automation of the assay for the routine determination of genotoxicities in mammalian cells. A contribution is provided in this respect, on the one hand, by the maximised fluorescence intensity of the AFP reporter and, on the other hand, the determination of as many repair responses to DNA damage as possible. In addition, in the case of all the cell types used for analysis so far, the possibility of suspension culture facilitates cell cultivation on a large scale (Moore, G. J. and Matsoukas, J. M. (1985) Bioscience Reports 5: 407-416) as well as apoptosis insensitivity with simultaneously high rates of DNA rearrangement (Mahdi, T., D. et al. (1998) Biol. Chem. 90: 15-27).

The assay, according to the invention, for the determination of genotoxicities by way of the detection of DNA rearrangements can also be transferred to transgenic mice. In the animal model, recombination can be detected only via genes whose products trigger a staining or fluorescence but not with a resistance gene, as used in most of the existing genotoxicity detection systems. Since the extremely strong CMV promoter was positioned in front of the acceptor substrate derived from the EGFP gene in each case, it must be expected, on the basis of existing studies, that, using the optimised constructs (FIGS. 5h to j), it is possible to detect autofluorescences and consequently recombination events in situ qualitatively by fluorescence microscopy and quantitatively by laser scanning cytometry (Compucyte) (Walter, I. et al. (2000) Histochemical Journal 32: 99-103). This means that tissue sections can be analysed directly for autofluorescences and consequently directly for DNA rearrangements without staining. To produce the corresponding transgenic mice, the test constructs need to be injected in the plasmidal form by microinjection into the pronucleus of fertilised oocytes. Following reimplantation into pseudo-pregnant nursing mice, transgenic offspring with intact integrates need to be identified by PCR analyses and Southern blots of genomic DNA from mice tails. Finally, the possibility is provided as a result of the retroviral vector design used throughout, of introducing anyone of the described constructs via retroviral transduction into the cells of murine embryos. Using this method, Jänisch and colleagues generated transgenic mice as early as in 1984 (Stuhlmann, H. et al. (1984) Proc. Natl. Acad. Sci. USA 81: 7151-7155), in which a bacterial transgene was active in many somatic tissues. For this purpose also, it is of great advantage that CMV promoters are introduced downstream of the recombination markers, i.e. the danger of the promoters in the transgenic animals being “switched off” by DNA methylation, which has been observed for the retroviral LTRs in the cells of the germ line, is reduced (Jähner, D. et al. (1985) Proc. Natl. Acad. Sci. USA 82: 6927-6931). By means of this invention, the prerequisites have thus been fulfilled for providing the answer to questions regarding the biotransformation of xenobiotics and the toxicology in specific tissues or for certain cell types. The successful establishment of a mouse model, moreover, fulfills the prerequisites for analysing the influence of certain endogenous factors (after crossings with knockout or transgenic mice) in order to detect changes in the gene expression profile connected with DSB repair (microarray technology). In this way, genetically caused sensitivities or resistances to certain noxae can be identified. This information can be used to develop individual chemotherapy protocols for genotyped patients.

The invention will be explained in the following by way of illustrations, examples and a sequence protocol.

### DESCRIPTION OF THE ILLUSTRATIONS (FIG.S)

FIG. 1: Principle of the recombination test system based on the reconstitution of an AFP gene. The simplest vector design for analysing conservative homologous recombination processes is illustrated. The vector backbone is formed by the retroviral vector p5NM from Dr. Carol Stocking, Heinrich-Pette-Institut Hamburg, which is derived from the MPSV retrovirus (5′LTR, 3′LTR and fully drawn black line) (Laker, C. et al. (1998) J. Virol. 72: 339-348). The puromycin resistance gene (Puro) is transcribed from the 5′LTR promoter and the encoded puromycin-N-acetyl transferase is expressed in fusion with an EGFP protein mutated in the chromophore region (_{Δ}EGFP). The 45 base pairs deleted in _{Δ}EGFP were replaced by an EcoRI and an I-SceI recognition sequence. The coding sequences for the I-SceI meganuclease (I-SceI) are transcribed, starting out from the Gal4 recognition sequences (GREs) by means of the estradiol-inducible transcription factors Gal4ER-VP. The N′-EGFP gene shortened in the 3′ portion of the EGFP gene is not preceded by a promoter. The administration of estradiol activates the GalER-VP transcription factor as a result of which the I-SceI meganuclease is expressed. The I-SceI meganuclease cleaves the DNA with a staggered cut within its recognition sequence in the _{Δ}EGFP gene. This initiates DSB repair processes which, by unilateral homologous DNA exchange between the N′-EGFP and the _{Δ}EGFP gene, can lead to the reconstitution of a wild-type EGFP gene. Grey, angular arrows indicate the orientation of the promoters, black arrows the orientation of the translated sequences. The I-SceI meganuclease was represented by way of a scissor symbol.

FIG. 2: Estradiol-inducible expression of I-SceI meganuclease.

KMV cells express the transcription factor GalER-VP which can be activated by estradiol and which consists of the DNA binding domain from the Gal4 transcription factor (Gal4), the estradiol binding domain of the human estradiol receptor (ER), and the transactivation domain of the Herpes virus protein VP16 (VP16). KMV cells were electroporated with vector pGC-Sce which contains 4 synthetic Gal4 recognition sequences as promoter upstream of the sequence coding for I-SceI. Following the selection of individual clones with an I-SceI expression cassette stably integrated into the genome, these were cultivated in the presence of 2 μM β-estradiol for 24 h. Estradiol-dependent expression of the I-SceI meganuclease in fusion with the peptide epitope of the anti-haemagglutinin (HA) antibody 12CA5 is shown for a representative clone in the Western blot. KMV cells which were transiently electroporated with pCMV-Sce, a plasmid constitutively expressing I-SceI, were used as positive control.

FIG. 3: Measuring recombination frequencies after targeted introduction of a double strand break.

By means of electroporation, a recombination substrate was introduced into KMV cells which substrate consists of a p5NM vector backbone with 5′LTR and 3′LTR, a puromycin resistance gene (Puro), a CMV promoter linked to the _{Δ}EGFP gene as the recombination acceptor in series, an N′-EGFP gene as the donor, and an I-SceI expression cassette inducible by estradiol. In the experiment shown, the cells were cultivated for 48 h in the presence of estradiol and green fluorescent cells were evaluated under the FACS scan flow cytometer. Intensively green fluorescent cells appear in the dot plot of green versus orange fluorescence (FL2 versus EGFP) as signals on the left hand side above the diagonal, whereas the signals for endogenous orange fluorescence of the cells are below the diagonal. Following excitation at 488 nm, 4% of the cells were green fluorescent whereas no fluorescent cells were observed in the negative controls for constructs without recombination acceptor or donor. In the positive control with wild-type EGFP expression, 40% fluorescent cells were detected. This corresponds to the transfection efficiency in the experiment.

FIG. 4: Mutations in the recombination acceptor substrates _{Δ}EGFP, EGFP-HR, and EGFP-EJ for the detection of homologous recombination events and end joining.

The sequences modified in the _{Δ}EGFP and EGFP-HR genes, in comparison with the wild-type EGFP gene between base pairs 163 and 220 of the coding region, are shown above the recombination construct from FIG. 1. The sequences modified in the EGFP-EJ gene in comparison with the wild-type EGFP gene are shown below the recombination construct. After introducing a targeted DSB into the synthetically introduced I-SceI recognition sequence, _{Δ}EGFP or EGFP-HR supports the homologous DNA exchange with the donor substrate, with EGFP-EJ in addition supporting also end joining by means of the microhomologies in the duplicated CCTAC-sequence. _{Δ}EGFP contains, 5′ to the new I-SceI recognition sequence, a recognition sequence for the restriction enzyme EcoRI (GAATTC). The grey circle illustrates another base modification in EGFP-HR in comparison with the wild-type EGFP gene. The principle of homologous recombination or non-homologous end joinings and the factors involved therein are illustrated schematically.

FIG. 5: Optimisation of the constructs for identifying genetic changes as genotoxicity indicators.

Starting out with the recombination test system in FIG. 1, all changes to the vector design to increase the fluorescence intensity (FI), the recombinative exchange rates (rate), and the spectrum of detectable genetic alterations (type) and consequently the sensitivity of the genotoxicity measurement as described in chapter 3, are summarised schematically. The legend regarding the individual sequence elements is given only for the first construct each and can be explained as follows: puromycin resistance gene (Puro), GalER-VP responsive promoter (GREs), coding region for the I-SceI meganuclease (I-SceI), EGFP gene shortened in the 3′ portion (N′-EGFP), CMV promoter (CMV), wild-type EGFP gene (EGFP), hygromycin resistance gene (hygro), SV40 promoter (SV40), EGFP gene with nonsense mutation in the start codon (met->stop), DsRed-Gen (Red), vector backbone of the murine Moloney leukemia virus (MoMuLV), transactivator controlled reversely by tetracyclin (rtTA), rtTA responsive promoter (TRE). The designations ΔEGFP/HR/EJ indicate that the construct illustrated was created in each case in the form of the three variants with either _{Δ}EGFP, EGFP-HR, or EGFP-EJ at the position indicated. Some types of genetic alterations were abbreviated, namely kHR for conservative homologous recombination, EJ for end joining, nkHR for non-conservative homologous recombination. Arrows indicate the orientation of the coding sequences.

### 4. ADVANTAGES OF THE INVENTION

This novel test system for the determination of genotoxicities overcomes all the disadvantages of previous detection systems: DNA damage and detection take place directly in the mammalian cells and not in the microorganisms. The detection system is transferable to living animals. The spectrum of possible DNA damage by mutagens is as wide as possible as a result of the detection of the most diverse range of recombinative repair processes and of point mutations and, in particular, also of every type of otherwise gene-inactivating events. The sensitivity of the system was maximised by the optimised use of strongly autofluorescent proteins as signals and the generation of long sequence homologies. The simple and rapid execution of the test fulfills the prerequisite for routine-type analyses on a large scale.

### 5. EXAMPLES

### Example 1

### Cloning of p5-Puro-CMV-(N′-EGFP)-CMV-Red-(EGFP-EJ)

Cloning of the construct p5-Puro-CMV-(N′-EGFP)-CMV-Red-(EGFP-EJ) will be described in the following in a manner representative of the vectors in FIG. 5 (compare FIG. 5i and chapter 6 sequence protocol). The retroviral vector p5NM from Dr. Carol Stocking, Heinrich-Pette-Institut Hamburg, which belongs to the family of MPSV vectors, was used as the basis (Laker, C. et al. (1987) Proc. Natl. Acad. Sci. USA 84: 8458-8462). Restriction enzymes, Rapid DNA Ligation Kit®, Klenow enzyme, T4 polymerase and shrimp alkaline phosphatase (SAP) were purchased from New England Biolabs, Schwalbach/Taunus, Roche, Mannheim, and MBI Fermentas, St. Leon-Rot. Bacteria transformations for cloning purposes were carried out using the bacteria strains XL10Gold®, SURE®, or SCS110 from Stratagene, Heidelberg, using standard methods (Ausubel, F. M. et al. (2000) Current Protocols in Molecular Biology, John Wiley and Sons). DNA blunt end formation with Klenow enzyme or T4 polymerase and DNA gel electrophoresis were also carried out according to these standard methods. Plasmid preparations from bacterial extracts were carried out in line with the instructions for the Qiaprep Miniprep Kit from Qiagen, Hilden, or with the Nucleobond® AX Kit from Macherey-Nagel, Düren, according to the manufacturer's instructions. The dephosphorylation of DNA ends by means of SAP was carried out according to the instructions of Roche, Mannheim. DNA fragments were purified after agarose gel electrophoresis using the QIAEX II Gel Extraction Kits from Qiagen, Hilden.

To prepare the the final cloning steps within the retroviral vector p5NM, a fragment was first produced from the expression cassette for the DsRed gene. For this purpose, the vector pDsRed1-N1 (Clontech, Palo Alto, Calif., USA) was cleaved by the restriction enzyme HindIII and AgeI, the DNA ends were end-filled by means of Klenow enzyme and subsequently religated with the Rapid DNA Ligation Kits in order to remove the multiple cloning site between the CMV promoter and the DsRed gene. In order to suppress the methylation of the plasmidal DNA in bacteria and to permit subsequent cleavage also at the XbaI recognition sequence which is unique in pDsRed1-N1, the plasmid DNA was amplified in the methylation-deficient bacterial strain SCS110 (Stratagene). The unmethylated plasmid was digested with the restriction enzyme AseI, the ends were end-filled by means of Klenow enzyme, and the fragment consisting of the CMV promoter and the downstream DsRed gene was excised by XbaI. The fragment was separated from the remaining vector sequences by means of the QIAEX II Gel Extraction Kits from Qiagen, Hilden. After isolation, the fragment was sub-cloned into the vector pUC18 (MBI Fermentas, St. Leon-Rot) after vector digestion with the enzymes SmaI and XbaI and dephosphorylation of both ends by means of SAP. The resulting vector pUC-CMV-Red was digested with the restriction enzyme NotI, treated with Klenow enzyme, re-digested with XbaI, and dephosphorylated. The fragment pCMV-(EGFP-EJ) was excised from pCMV-(EGFP-EJ) (compare example 2), by cleavage with XhoI and Klenow enzyme treatment, re-digestion with XbaI, and ligation with the NotI/XbaI-cleaved pUC-CMV-Red. pCMV-(EGFP-EJ) is pEGFP-N1 from Clontech, in which the sequence coding for chromophore amino acids of the EGFP gene had been mutated in such a way that an I-SceI recognition sequence and short sequence repeats were created 5′ and 3′ to this recognition sequence, so that not only homologous recombination with a second EGFP gene variant but also end joining can take place via these repeats after I-SceI digestion. The pUC-CMV-Red-(EGFP-EJ) plasmid resulting from the ligation, in which DsRed and EGFP-EJ had same orientation, was cut by EcoRI and SalI. The CMV-Red-(EGFP-EJ) fragment was ligated with the correspondingly digested and dephosphorylated p5NM vector, to give p5-CMV-Red-(EGFP-EJ).

In parallel, the puromycin resistance gene from the Clontech vector pRetroOn was cloned via the enzymes NaeI and EcoRI into pUC18 following SmaI and EcoRI restriction enzyme digestion (pUC-Puro). Subsequently, the ΔEGFP-PCR fragment (compare example 2) was cloned in frame adjacent to the puromycin resistance gene via the restriction enzyme recognition sequences SexAI and EcoRI in the pUC-Puro vector (pUC-Puro-ΔEGFP). In the next step, the EcoRI recognition sequences introduced artificially via PCR into _{Δ}EGFP were used at the 3′ end and in the central mutated region to excise the 3′ half of the gene. This fragment was cloned via the EcoRI recognition sequence downstream of the puromycin resistance gene in a pUC-Puro vector such that the puromycin resistance gene and the _{Δ}EGFP half gene had the same orientation. The resulting pUC-Puro-_{Δ}EGFP1/2 vector now contained one artificial NotI recognition sequence at the 3′ end of the _{Δ}EGFP half gene. pUC-Puro-ΔEGFP1/2 was cleaved with NsiI and the DNA ends were end-filled by T4 polymerase treatment. Subsequently, pUC-Puro-_{Δ}EGFP1/2 was freed of the 3′ half of the _{Δ}EGFP gene by re-digestion at the synthetic NotI restriction recognition sequence and replaced by a CMV-(EGFP-EJ) fragment. For this purpose, the vector pCMV-(EGFP-EJ) (compare example 2) was cleaved with AseI, the DNA ends were end-filled with Klenow enzyme, re-digested with NotI, and ligated with the pUC-Puro vector fragment. In this way, the vector pUC-Puro-CMV-(EGFP-EJ) was formed.

The EGFP gene fragment N′-EGFP shortened at the 3′ end was excised from pEGFP-N1 via restriction digestions with GsuI and AgeI. N′-EGFP lacks 277 base pairs at the 3′ end of the coding region of the EGFP gene. After blunt end formation via T4 polymerase, the N′-EGFP fragment was ligated into the multiple cloning site of vector p5NM, after vector cleavage with HindIII, blunt end formation by means of Klenow enzyme, and dephosphorylation (p5NM-(N′-EGFP)).

In the plasmid pUC-Puro-CMV-(EGFP-EJ) the region coding for EGFP-EJ was removed via NotI and XhoI restriction digestion, the remaining ends were end-filled with Klenow enzyme and dephosphorylated. The N′EGFP gene was inserted in the same orientation as the original EGFP-EJ gene by excision via EcoRI and SalI digestion from p5NM-(N′-EGFP), end-filled with Klenow enzyme, and ligated with the pUC-Puro-CMV vector fragment. From the resulting vector pUC-Puro-CMV-N′EGFP finally, the fragment Puro-CMV-N′EGFP was inserted, as BamHI fragment, in the vector p5-CMV-Red-(EGFP-EJ), which was constructed in the beginning, following its BamHI digestion and dephosphorylation (both EGFP gene variants in the same orientation). In this way, the final construct p5-Puro-CMV-(N′-EGFP)-CMV-Red-(EGFP-EJ) was produced. After cloning, the recombination construct was sequenced in the segments which cannot be checked via antibiotic resistance or red autofluorescence: N′-EGFP with primers EGFPwt-1 and EGFPSeq1, EGFP-EJ with primer EGFPSeq3 and the CMV promoter preceding N′EGFP. For sequence analysis, the ABI PRISM™ Big Dye Ready Reaction Cycle Sequencing Kit from PE Applied Biosystems, Weiterstadt, was used according to the manufacturer's instructions. Electrophoretic separation and automatic analysis of the absorption maxima was carried out in an ABi 377-96 automatic sequencing facility with corresponding Basecaller software.

Sequencing Primers:

| | |
|---|---|
| EGFPwt-1: | GTGACCACCCTGACCTAC |
| EGFPSeq1: | GCAGCACGACTTCTTCAAGT |
| EGFPSeq3: | GTTGTGGCTGTTGTAGTTGTA |

The plasmid “p5-Puro-CMV-(N′-EGFP)-CMV-Red-(EGFP-EJ)” was registered on Nov. 14, 2000, at the Deutsche Sammlung von Mikroorganismen and Zellkulturen (DSMZ), Mascheror Weg 1b, 38124 Brunswick, Germany, denoted by number DSM 13848 in line with the Budapest convention.

### Example 2

### Generation of the _{Δ}EGFP and EGFP-EJ Genes by PCR Mutagenesis

In a manner representative of the different mutations in the sequence coding for the chromophore amino acids within the EGFP gene, the construction of the _{Δ}EGFP gene and the EGFP-EJ gene will be described here.

_{Δ}EGFP was constructed via stepwise PCR amplifications. The terminal PCR primers (EGFP-13 and −15) for the complete _{Δ}EGFP-PCR fragment introduced synthetic restriction enzyme recognition sites into the ΔEGFP gene, namely for SexAI at the 5′ end and for BamHI and EcoRI at the 3′ end, so that these restriction sites were available for subsequent cloning in the reading frame of the puromycin resistance gene in pUC-Puro (compare example 1). The mutated region at the chromophore amino acids was engineered by firstly producing the sequence portion 5′ and 3′ to the mutation in the ΔEGFP gene via 2 independent PCR reactions. The desired PCR products were purified after agarose gel electrophoresis using the QIAEX kit. Subsequently, the two PCR fragments were fused at the synthetic ends covering the mutation via a ligating PCR. In ΔEGFP the base pairs coding for the chromophore amino acids 65-67 of the EGFP gene as well as 29 additional base pairs 5′ and 7 base pairs 3′ to it are thus replaced by the recognition sequences for the meganuclease I-SceI and the restriction endonuclease EcoRI (compare FIG. 4).

Reaction Mixture 1:

100 ng pEGFP-N1

50 pmol primer EGFP-14

50 pmol primer EGFP-15

200 μM dNTP

10 μl 10× Taq polymerase buffer (Qiagen)

10 μl Q solution (Qiagen)

1 μl Taq DNA polymerase (Qiagen)

ad 100 μl H₂O

Reaction mixture 2:

Like reaction mixture 1 except for the primers:

50 pmol primer EGFP-11

50 pmol primer EGFP-13

Reaction Mixture 3 (Ligating):

Purified PCR products from reactions 1 and 2

50 pmol primer EGFP-13

50 pmol primer EGFP-15

200 μM dNTP

10 μl 10× Taq polymerase buffer (Qiagen)

20 μl Q solution (Qiagen)

1 μl Taq polymerase (Qiagen)

ad 100 μl H₂O

The reaction mixture was firstly incubated at 92° C. for 4 min each. Subsequently, 35 cycles were carried out under the following conditions:

92° C. 60 s

60° C. 60 s

72° C. 120 s

Subsequently, the reaction mixture was incubated at 72° C. for 4 min.

The _{Δ}EGFP-PCR fragment thus formed was inserted, after restriction digestion with SexAI and EcoRI, in the plasmid pUC-Puro after corresponding digestion and dephosphorylation (compare example 1).

EGFP-EJ was constructed like _{Δ}EGFP via stepwise PCR reactions. EGFP-EJ contains an I-SceI recognition sequence as insert, in the EGFP sequence location which codes for chromophore amino acids (compare FIG. 4). In addition, 5 base pairs of the EGFP sequence were duplicated so that homologies of 5 base pairs in length exist 5′ and 3′ to the I-SceI recognition sequence. Following the introduction of a DSB by I-SceI, these short homologies are used in EJ for the reconstitution of the wild-type EGP sequence.

In contrast to the construction of _{Δ}EGFP, the primer pairs EGFP-EJ-1 and EGFP-13 or EGFP-EJ-2B and EGFP-15 were used in the reaction mixtures 1 and 2. The generated EGFP-EJ-PCR fragment was inserted, following QIAEX purification and restriction digestion with SexAI and EcoRI, into the plasmid pUC-Puro after corresponding digestion and dephosphorylation (compare example 1). To produce pCMV-(EGFP-EJ) (compare example 1), the resulting vector pUC-Puro-(EGFP-EJ) was digested with SexAI, the DNA ends were filled in with Klenow enzyme and the EGFP-EJ fragment was obtained via NotI re-digestion. The purified fragment was inserted into the plasmid pEGFP-N1, in place of and in the same orientation as the EGFP gene, after cleavage with HindIII, treatment with Klenow enzyme, and re-digestion with NotI. The plasmid pCMV-(EGFP-EJ), resulting from the ligation, was used for further cloning and, where necessary, was amplified in the methylation deficient bacterial strain SCS110 (compare example 1).

All DNA fragments generated by PCR were sequenced following cloning (compare example 1).

Primer:

| | |
|---|---|
| EGFP-11: | GTTCATCTGCACCACCGGCAAGCTGCCCGGAATTCTAGGGATAACA |
| | GGGTAATGCTTCAGCCGCTACCCCGAC |
| EGFP-13: | AGGAGGGAATTCGGATCCGCGGCCGCTTTACTTGTACAGCT |
| EGFP-14: | TCCCTAGAATTCCGGGCAGCTTGCCGGTGGTGC |
| EGFP-15: | CGCGCGACCTGGTGCATGACCCGCAAGCCCGGTGCCATGGTGAGC |
| | AAGGGCGAGGAGC |
| EGFP-EJ-1: | TGACCTACTAGGGATAACAGGGTAATCCTACGGCGTG |
| EGFP-EJ-2B: | CACGCCGTAGGATTACCCTGTTATCCCTAGTAGGTCAGGGTGGTCACGA |

### Example 3

### Analysis of Individual KMV Cell Clones with a Genomic pGC-Sce Integrate Following I-SceI Expression After Estradiol Administration.

KMV cells represent K562 leukemia cells (Andersson, L. C. et al. (1979) Int. J. Cancer 23: 143-147) which express the transcription factor GalER-VP which can be activated by the administration of estradiol (Braselmann, S. et al. (1993) Proc. Natl. Acad. Sci. USA 90: 1657-1661). Following electroporation with the plasmid pGC-Sce, KMV clones were isolated with the construct integrated into genome (compare example 6). These clones were cultivated in tissue culture flasks from Nunc™, Wiesbaden. RPMI without phenol red (Gibco/BRL, Eggenstein) was used as the culture medium together with 10% FCS without steroid hormones (Promocell). The incubation of the cells was carried out at 37° C. and in 5% CO₂ in the incubator.

To activate GalER-VP and consequently indirectly the promoter in pGC-Sce via the Gal4 recognition sequences upstream of the sequence coding for I-SceI, 2 μM β-estradiol (ICN Biomedicals GmbH, Meckenheim) were initially included into the medium, though after the determination of the lowest concentration (50 nM) for complete promoter induction only 200 nM were used, and cultivated for 4-24 h. Subsequently, the cells were sedimented by centrifugation (1000×g, 5 min, room temperature), washed 1× with PBS (140 mM NaCl; 3 mM KCl; 8 mM Na₂HPO₄; 1,5 mM KH₂PO₄; pH 7,4), and 5×10⁶ cells each were suspended in 50 μl sample buffer (65 mM Tris/HCl, pH 6.8; 10% glycerine; 2.3% SDS; 5% β-mercaptoethanol (freshly added); 0.05% bromophenol blue) and boiled for 5 min. KMV cells, which had been electroporated transiently with pCMV-Sce, a constitutively, I-SceI-expressing plasmid (compare example 4), were used as positive control.

Subsequently, the proteins from 1-2×10⁶ cells were separated by discontinuous gel electrophoresis in a 15% SDS polyacrylamide gel (Lämmli, U.K. (1970) Nature 227: 680-685). A mixture of proteins with defined molecular weight was used as molecular weight standard (Sigma, Munich). The separation took place at a strength of the electric current of 25 mA/gel. Subsequently, the proteins were electrotransferred from the gel to a polyvinylidene fluoride membrane (Immobilon-P, Millipore) (Towbin, H. (1979) Proc. Natl. Acad. Sci. USA 76: 4350-4354). The transfer took place in a Hoefer wet blot apparatus filled with Tris-glycine buffer (192 mM glycine; 50 mM Tris/HCl, pH 8.3) with cooling on ice at 100 V for 60 min. Subsequently, the membrane was rinsed in TBS-T buffer (20 mM Tris/HCl, pH 7.6; 137 mM NaCl; 0.2% Tween 20) with 5% skimmed milk powder in order to saturate non-specific antibody binding sites. To detect the estradiol-dependent expression of the I-SceI meganuclease, the fact was exploited that it is expressed by the construct pGC-Sce fused to the peptide epitope of the anti-haemagglutinin (HA) antibody 12CA5. For this reason, the membrane was incubated in a suitable dilution of the antibody 12CA5 (1/20-1/50 fold dilution of the hybridoma cell culture supernatant) in TBS-T buffer with 5% skimmed milk powder overnight at 4° C. The membrane was then washed 4× for 15 min each, with TBS-T buffer, and incubated for 60 min with the secondary antibody (10⁻⁵ mg/ml) in TBS-T buffer with 5% skimmed milk powder. The membrane was then washed 3× for 15 min each, with TBS-T buffer. The secondary antibody which binds to the FC part of the primary antibody is conjugated with a peroxidase. This catalyses the chemiluminescence reaction for antigen detection, namely the oxidation of luminol by hydrogen peroxide. The excited luminol molecule emits light at a wavelength of λ=428 nm which leads to darkening of an X-ray film. This chemiluminescence reaction was triggered by means of a Super-Signal® ULTRA chemiluminescence solution from Pierce (Illinois, USA) according to the manufacturer's instructions, a Biomax-MR X-ray film was placed onto the drained membrane, and the film was developed after exposure.

### Example 4

### Measurement of Spontaneous, I-SceI-Expression Enhanced, or noxa-Induced Recombination Frequencies by EGFP Fluorescence Detection.

During the transient recombination assay, the DNA substrates were introduced into the cells by the physical transfection method of electroporation. During this process, the pores of the cell membrane are depolarised by the brief generation of an electrical field and thus made permeable for DNA. Since the substrates are not vectors that replicate episomally in mammals, they are diluted during the days following transfection, i.e. the assay is transient.

The electroporation of PA317, MethA, HL60, WTK1, K562 and derivative cells was carried out as described by Baum and colleagues (Baum, C. et al. (1994) Biotechniques 17: 1058-1062). 2-5×10⁶ PA317 or MethA or 10⁷ cells of the remaining cell types were harvested by trypsinisation (cells from adhesion culture) or by centrifugation (cells from suspension culture). The cell pellet was resuspended in 400 μl DMEM or RPMI medium with 10% FCS and mixed with 20 μg DNA in an electroporation cuvette (Biorad, Munich). Electroporation took place at 240 V and 1050 mF. Immediately afterwards, the electroporated cells were transferred into a cell culture dish or cell culture flask and incubated in the incubator (compare example 3).

If the cells or the recombination construct used permitted an I-SceI expression as a function of estradiol, the electroporated cells were split and, for the analysis of DNA rearrangements following the introduction of a DSB, 200 nM β-estradiol (ICN Biomedicals GmbH, Meckenheim) were introduced, immediately after electroporation, into the culture medium of one half of the cell batches. For cells or recombination constructs not offering this possibility, spontaneous recombination processes were distinguished from those following I-SceI digestion by electroporating the cells with a mixture of 10 μg DNA recombination substrate and 10 μg pBlueScript M13-DNA from Stratagene, San Diego, USA (spontaneously) or 10 μg DNA recombination substrate and 10 μg pCMV-Sce-DNA (I-SceI-induced). The plasmid pCMV-Sce permits the constitutive expression of the I-SceI meganuclease by means of the extremely strong CMV promoter in mammalian cells.

For the quantitative detection of green or red fluorescent cells, 2×10⁶ cells were harvested at different times (4-96 h following electroporation) by trypsinisation or by centrifugation, washed 1× with PBS including 0.2% EDTA and resuspended in 2 ml PBS with 0.2% EDTA. Subsequently, 30000 cells per sample were analysed in the Coulter Counter EPICS XL-MCL™ FACS scan flow cytometer or in the Calibur from Becton Dickinson. The quantitative evaluation of the fluorescent cells in comparison to the non-fluorescent ones was carried out by means of the SystemII™ software. Apart from FACS analysis, green or red fluorescent cells could also be visualised under the immunofluorescence microscope (Zeiss Axiovert 35) with the corresponding filter sets. The Compucyte laser scanning cytometer represents the optimum tool for quantifying fluorescent cells in a population of non-fluorescent cells since this device combines the advantages of individual cell analysis under the fluorescence microscope with sorting in the cytometer.

While measuring spontaneous recombination frequencies in cells with a chromosomally integrated recombination construct (compare example 6) by EGFP fluorescence detection, the cells remained either untreated or were electroporated with 10 μg pBlueScript M13-DNA. To determine the responses to certain noxae, the cells were treated with ionising radiation at a dose of 500 rad (¹³⁷Cs irradiation source) or with 100 μM etoposide (Sigma) in the medium for 2 hours. When measuring DNA rearrangements following I-SceI expression, the meganuclease was introduced into the cells by electroporation with 10 μg of the plasmid pCMV-Sce. Fluorescence analyses were carried out as in the transient assay.

### Example 5

### Generation of a Helper Cell Line Producing Retroviruses

Retroviral packaging cell lines provide the possibility of producing intact retrovirus particles from replication-defective virus genomes since the proteins required for replication and packaging (gag, pro, pol-env gene products) are produced by the packaging cell line (Coffin, J. M. (1996) in Fundamental Virology, Fields, B. N., Knipe, D. M. and Howley, P. M. (eds.), Lippincott-Raven Publishers, Philadelphia, 763-844). For packaging of retrovirus genomes in these packaging cell lines, retroviral vectors require a 5′LTR with a primer binding site, a 3′LTR, and a packaging signal. In addition, foreign sequences between the LTRs can be transduced via the resulting transgenic retroviruses up to an overall genome size of 10 kilobase pairs in length in total. Replication-deficient retroviruses from packaging cell lines are capable of infecting cells but not of generating new virions. To produce retroviruses for the transduction of the recombination constructs, the murine packaging cell line PA317 was used which is capable of producing amphotrophic retroviruses, i.e. it carries all the genes for packaging replication-deficient retroviruses which are capable of infecting the cells of mice, humans, chickens, dogs, and cats (Miller, A. D. and Buttimore, C. (1986) Mol. Cell. Biol. 6: 2895-2902).

PA317 cells were cultivated in adhesion culture in tissue culture dishes from Nunc™, Wiesbaden. The culture medium consisted of DMEM (Gibco/BRL, Eggenstein) with 10% FCS (Boehringer, Mannheim). The incubation of the cells took place at 37° C. and in 5% CO₂ in the incubator. For passaging, the cells were washed with trypsin solution (0,05% trypsin; 1×PBS; 0.5 mM EDTA, pH 8.0 from Gibco/BRL, Eggenstein) after sucking off the culture medium, incubated for approximately 5 min, and the detached cells were transferred into culture medium. The recombination constructs which had all been generated on the basis of retroviral vectors were introduced into PA317 cells by electroporation (electroporation method as in example 4). The constructs described as the preferred embodiment in this invention each carry an expression cassette for puromycin resistance which was used for the selection of transgenic clones. Following electroporation with the relevant construct, cells were cultivated for 2 days for recovery in normal medium. Subsequently, the culture medium was adjusted for 14 days to a puromycin concentration of 4.5 μg/ml, the medium being changed every 2-3 days while the majority of the cells died. After 14 days, it was possible to observe individual cell clones of at least 100 cells at the bottom of the cell culture dish. At this point, the medium was removed by suction and washed with trypsin solution. Subsequently, a sterile cloning cylinder of borosilicate glass was fixed around the desired clone by means of sterile silicone grease. Into this ring, 100 μl trypsin solution were introduced and the cells which had become detached were removed a few minutes later by suction via a pipette and cultivated each in a well of a 96 or 24 well plate. After proliferation of the cells, the integrity of the construct following random integration into the cellular genome was examined by PCR analysis (compare example 7). The release of infectious virus particles in the culture supernatant was tested for transgenic PA317 clones with the correct integrate by infecting murine MethA or human K562 cells and testing them for the formation of cell clones resistant to puromycin (compare example 6). After reaching a cell number of approximately 6×10⁶ cells, these were immediately frozen in liquid nitrogen for long-term storage since the virus titer decreases during prolonged cultivation.

### Example 6

### Infection of K562 Cells with Retroviruses and Isolation of Infected Clones

Since the K562 cells are cells which grow in suspension culture, the cells need to be concentrated before infection with retroviruses, in contrast to cells growing adherently. For this reason, the method of retroviral infection is described for K562 cells representatively.

For the infection, K562 cells were kept in culture in 20 ml of RPMI medium (compare example 3) until a total number of 1-2×10⁷ of exponentially growing cells was reached in a tissue culture flask with a base of 80 cm² (Nunc™, Wiesbaden). In parallel, cells of the PA317 clone were cultivated with the construct of interest integrated into the genome, directly after cloning or thawing (compare example 5). The medium was changed and removed 24 h later as virus supernatant and filtered to make it sterile (0.45 μm). After initial growth K562 cells were harvested by centrifugation (1000×g, 5 min, room temperature) and the cell pellet was resuspended in 10 ml virus supernatant for infection or in DMEM medium with 10% FCS as negative control. The mixture was incubated in the concentrated state in a tissue culture flask with a base of 25 cm² for 1 h and subsequently centrifuged for 1 h at 1000×g and room temperature in order to increase the rate of infection. Subsequently, the cells were incubated for 1 d and transferred into a tissue culture flask with a 80 cm² base.

It was possible to isolate successfully infected cells on the basis of the puromycin resistance gene transduced via the retrovirus, as individual cell clones in softagar containing puromycin. Cloning of individual cell clones in softagar was carried out in the same way for K562 and WFK1 cells following electroporation with DNA. For this purpose, the infected K562 cells were plated, after a 2 d recovery period in the tissue culture flask (100 ml), in softagar cultures (3 ml each) consisting of RPMI medium; 200 mM glutamine; 1% sodium pyruvate; 12% FCS; 0.33% Bactoagar (Difco, Detroit, USA) with puromycin (0.25 μg/ml for K562 and 0.1 μg/ml for WTK1). The cell count amounted to 1.5×10⁵ per well in a 6 well plate from Nunc™, Wiesbaden. After 10 days of incubation in the incubator, it was possible to isolate individual cell clones with a Pasteur pipette and to cultivate them in 96 or 24 well plates in liquid medium. After multiplication of the cells, the integrity of the provirus was examined by genomic PCR analysis (compare example 7).

### Example 7

### Verification of the Integrity of a Recombination Construct Following Stable Integration Into the Genome.

Following genome integration, the retroviral DNA, the so-called provirus, is flanked by the LTRs. The integration into the genome is a random integration, but normally takes place with the order of the genes being retained. However, in order to be able to exclude rearrangements in the course of the retroviral infection cycle, the proviruses in infected cells, which exhibit a resistance to puromycin, were analysed regarding the integrity of the recombination marker. This control was necessary in particular for the selection of the potentially retrovirus producing PA317 clones in order to ensure that the sequences to be transduced via retroviruses remain unchanged after the electroporation of the PA317 cells and subsequent random integration of the construct concerned into the genome.

Genomic DNA was obtained for each cell type from approximately 5×10⁶ cells according to the instructions of the manufacturer of the QIAamp DNA Mini Kit, Qiagen, Hilden. Applying a constant amount equivalent to a volume of 2 μl of the genomic DNA isolate, PCR reactions were carried out to detect the different sequence segments with a length of 400-1000 base pairs of the complete construct. In parallel, a reaction with 10-100 ng of the construct in the plasmidal form was carried out as positive control. A reaction without primer and a reaction without template DNA were carried out as negative controls.

Reaction Mixture 1 (for the Detection of the Sequences Between the Puromycin Resistance Gene and the EGFP Gene):

DNA template (compare text above)

50 pmol primer Puro1

50 pmol primer EGFP20-2

200 μM dNTP

10 μl 10× Taq polymerase buffer (Qiagen)

20 μl Q solution (Qiagen)

1 U Taq DNA polymerase (Qiagen)

ad 100 μl H₂O

Reaction Mixture 2 (for the Detection of the Sequences Between the I-SceI Gene and the EGFP Gene):

Same as for reaction mixture 1 except for the primers:

50 pmol primer Sce1-1 or Sce1-2b

50 pmol primer EGFP20-2

Reaction Mixture 3 (for the Detection of the Sequences in the EGFP Gene):

Same as for reaction mixture 1 except for the primers:

50 pmol primer EGFP-PCR1

50 pmol primer EGFP-PCR2 or EGFP-PCR3

Reaction Mixture 4 (for the Detection of the Sequences in the Hygromycin Resistance Gene):

Same as for reaction mixture 1 except for the primers:

50 pmol primer Hyg1-1

50 pmol primer Hyg2-2

Reaction Mixture 5 (for the Detection of the Sequences Between the Hygromycin Resistance Gene and the EGFP Gene):

Same as for reaction mixture 1 except for the primers:

50 pmol primer Hyg4-1

50 pmol primer EGFP-PCR2

The reaction mixture was, in each case, incubated initially at 96° C. for 5 min. Subsequently, 35 cycles were carried out under the following conditions:

94° C. 60 s

60° C. 60 s

72° C. 120 s

Subsequently, the reaction mixture was incubated at 72° C. for 7 min.

The PCR fragments formed were analysed by agarose gel electrotrophoresis next to the Lambda DNA/HindIII ladder from MBI-Fermentas, St. Leon-Rot, for the determination of size and quantity.

Primers:

| | |
|---|---|
| Puro1: | CCCGCAACCTCCCCTTCTAC |
| EGFP20-2: | GTGAACAGCTCCTCGCCCTTG |
| Sce1-1: | GGTCCGAACTCTAAACTGCTGA |
| Sce1-2b: | TCGGGGTCAGGTAGTTTTCA |
| EGFP-PCR1: | GTGAGCAAGGGCGAGGAGCT |
| EGFP-PCR2: | CTTTACTTGTACAGCTCGTCCAT |
| EGFP-PCR3: | GACGTTGTGGCTGTTGTAGTTGTA |
| Hyg1-1: | GGAAAGCCTGAACTCACCGCGA |
| Hyg2-2: | GCTTCTGCGGGCGATTTGTGTA |
| Hyg4-1: | TACACAAATCGCCCGCAGAAGC |

1. Vector which exhibits the general structure
5′-[Ins1]-[Prom1]-[Marker1]-[Prom2]-[Ins2]-[Marker2]-3′
wherein
[Ins1] is a sequence segment which codes for a selection marker, for a transcription factor controlled by tetracyclin or estradiol, for a protein to be analysed with respect to repair, genome stability, genotoxicity, or cancer susceptibility, for an autofluorescent protein (AFP), for a bioluminescent enzyme or an enzyme that converts chemiluminescent substrates (LE) or a mutant thereof, or which can be altogether absent
[Prom1] is a promoter or can be altogether absent,
[Marker1] is a sequence segment which is any desired DNA fragment with a homology of at least approximately 200 bp to the sequence segment of [Marker2] or which codes for a derivative or a mutant of an autofluorescent protein (AFP) or a bioluminescent enzyme or an enzyme that converts chemoluminescent substrates (LE),
[Prom2] is a promoter, a spacer, or may be absent altogether,
[Ins2] is a sequence segment which codes for a selection marker, a transcription factor controlled by tetracyclin or estradiol, for an AFP, LE, or a mutant thereof, for I-SceI, or for a protein to be analysed with respect to repair, genome stability, genotoxicity, or cancer susceptibility or a spacer,
[Marker2] is a sequence segment which is any desired DNA segment with a homology of at least approximately 200 bp to the sequence segment of [Marker1] or which codes for a derivative or a mutant of an autofluorescent protein (AFP) or a bioluminescent enzyme or an enzyme that converts chemoluminescent substrates (LE),
wherein [Marker1] and [Marker2] are two homologous DNA sequence segments which trigger the alteration of the gene within the sequence segment in [Ins2] by DNA exchange.

2. Vector according to claim 1, characterised in that the wild-type gene (AFP or LE) is the nucleic acid sequence which codes for the enhanced green fluorescent protein (EGFP), for the humanized renilla green fluorescent protein (hrGFP), for the enhanced blue fluorescent protein (EBFP), for the enhanced cyan fluorescent protein (ECFP), for the enhanced yellow fluorescent protein (EYFP), or for the red fluorescent protein (RFP or DsRed) or a nucleic acid sequence which codes for an enzyme whose activity is detectable via a chemoluminescence reaction.

3. Vector according to claims 1 or 2, characterised in that [Marker1] and [Marker2] are two random sequence segments homologous, however, over at least 200 bp, wherein a loss or modification of [Ins2] occurs in the case of non-conservative homologous recombination or replication slippage between [Marker1] and [Marker2], inactivation of the gene product occurrs in the case of inactivating mutations in [Ins1] or [Ins2], the activation of the gene product concerned occurrs in the case of reverse mutations in [Ins1] or [Ins2], and the wild-type gene is reconstituted by homologous DNA exchange if [Marker1] and [Marker2] are sequence segments which code for an autofluorescent protein, for a bioluminescent enzyme, or for an enzyme that converts chemiluminescent substrates or for a mutant thereof.

4. Vector according to claim 1 or 2, characterised in that [Marker1] and [Marker2] are two random DNA sequence segments homologous, however, over at least approximately 200 bp.

5. Vector according to claims 1 to 4, characterised in that [Ins1] is a sequence segment which contains a selection marker gene.

6. Vector according to claims 1 to 4, characterised in that [Ins1] is a sequence segment which contains a nucleic acid sequence which codes for an autofluorescent protein, for a bioluminescent enzyme, or for an enzyme that converts chemiluminescent substrates or for a mutant thereof.

7. Vector according to claims 1 to 6 characterised in that [Ins2] is a sequence segments which contains a selection marker gene.

8. Vector according to claims 1 to 6 characterised in that [Ins2] is a sequence segment which contains a nucleic acid sequence which codes for an autofluorescent protein or for a bioluminescent enzyme, or an enzyme that converts chemiluminescent substrates or for a mutant thereof.

9. Vector according to claim 1 to 8 characterised in that a recognition sequence for meganuclease I-SceI from Saccharomyces cerevisiae is present on the vector.

10. Vector according to claim 1 to 3 characterised in that [Marker1] and [Marker2] are sequence segments which contain a nucleic acid sequence which codes for an autofluorescent protein, for a bioluminescent enzyme or an enzyme that converts chemiluminescent substrates, or for a mutant thereof, the wild-type gene being reconstituted by homologous DNA exchange.

11. Vector according to claim 10, characterised in that [Ins1] is a sequence segment which contains a selection marker gene.

12. Vector according to claim 10, characterised in that [Ins1] is a sequence segment which contains a nucleic acid sequence which codes for an autofluorescent protein or for a bioluminescent enzyme; or an enzyme that converts chemiluminescent substrates or for a mutant thereof.

13. Vector according to claims 10 to 12 characterised in that [Ins2] is a sequence segment which contains a selection marker gene.

14. Vector according to claims 10 to 12 characterised in that [Ins2] is a sequence segment which contains a nucleic acid sequence which codes for an autofluorescent protein, for a bioluminescent enzyme, or for an enzyme that converts chemiluminescent substrates or for a mutant thereof.

15. Vector according to claims 1 to 3, characterised in that the transcription factor controlled by tetracyclin or estradiol is the transactivator rtTA reversely controlled by tetracyclin or the GalER-VP activated by estradiol.

16. Vector according to claims 1 to 3, characterised in that the protein to be analysed with respect to repair, genome stability, genotoxicity, or cancer susceptibility is the repair surveillance factor p53.

17. Vector according to claims 10 to 13, characterised in that [Ins2] is a spacer with a length of approximately 1 kb.

18. Vector according to claims 10 to 17, characterised in that [Prom1] is a CMV promoter or an alternative eukaryotic promoter by means of which at least as high an AFP expression is achieved as when a CMV promoter is used.

19. Vector according to claims 1 to 18, characterised in that [Prom2] is a promoter from the group consisting of an estradiol responsive (GRE) or a tetracyclin-responsive (TRE) promoter, the SV40 promoter, the CMV promoter, or a promotor effecting an expression similarly high in various cell and tissue types as the CMV promoter, the latter for AFP genes contained in the sequence segment [Ins2].

20. Vector according to claims 1 to 18, characterised in that [Prom2] is a spacer with a length of approximately 1 kb.

21. Vector according to claims 1 to 20 characterised in that the autofluorescent protein is selected from the group consisting of humanized renilla green fluorescent protein (hrGFP), enhanced green fluorescent protein (EGFP), enhanced blue fluorescent protein (EBFP), enhanced cyan fluorescent protein (ECFP), enhanced yellow fluorescent protein (EYFP), red fluorescent protein (RFP or DsRed), or variants or mutants of these proteins.

22. Vector according to claims 1 to 20 characterised in that the bioluminescent enzyme or the enzyme that converts chemoluminescent substrates is alkaline phosphatase, β-galactosidase, peroxidase, luciferase, or a variant or mutant thereof.

23. Vector according to claims 1 to 10 characterised in that [Marker1] and [Marker2] each contain at least one mutation, the positions of the mutations being selected such that
a) the section of the sequence between the position in [Marker2] which corresponds to a mutation in [Marker1] and the first mutation in [Marker2] which is situated in the 3′ direction, or
b) the section of the sequence between the position in [Marker1] which corresponds to a mutation in [Marker2] and the first mutation in [Marker1] which is situated in the 3′ direction
comprises at least approximately 150 base pairs.

24. Vector according to claims 1 to 23, characterised in that the sequence segment [Prom2] is deleted and the sequence segment [Ins2] immediately starts at the 3′ end of the sequence segment [Marker1].

25. Vector according to claims 1 to 24 characterised in that the sequence segment [Prom1] is deleted and the sequence segment [Marker1] immediately starts at the 3′ end of the sequence segment [Ins1].

26. Vector characterised in that it is derived from a vector according to claims 10 to 24 by the sequence segments [[Prom1]-[Marker1]] and [Marker2] being exchanged in their positions whereby, the orientation of the two sequence segments is simultaneously reversed.

27. Vector characterised in that it is derived from a vector according to claims 10 to 23 by the orientation of the sequence segments [[Prom1]-[Marker1]] and/or [Marker2] being reversed or the sequence segments being exchanged without a change in orientation and the vector exhibits the sequence
5′-[Ins1]-(3′-[Marker1]-[Prom1]-5′)-[Prom2]-[Ins2]-[Marker2]-3′,
5′-[Ins1]-[Prom1]-[Marker1]-[Prom2]-[Ins2]-(3′-[Marker2]-5′)-3′,
5′-[Ins1]-(3′-[Marker1]-[Prom1]-5′)-[Prom2]-[Ins2]-(3′-[Marker2]-5′)-3′
or 5′-[Ins1]-[Marker2]-[Prom2]-[Ins2]-[Prom1]-[Marker1]-3′

28. Vector characterised in that it is derived from a vector according to claims 1 to 23 by the orientation of the sequence segment [[Prom2]-[Ins2]] being reversed.

29. Vector characterised in that it is derived from a vector according to claims 1 to 24 by the sequence segment [Marker2] being deleted.

30. Vector according to claims 1 to 29 characterised in that [Marker1] within the sequence segment additionally contains a recognition sequence for meganuclease I-SceI from Saccharomyces cerevisiae or is adjacent to it.

31. Vector, characterised in that it is derived from a vector according to claims 1 to 30 in which [Ins2] is a transcription factor controlled by tetracyclin or estradiol, by insertions between the 3′ end of the sequence segment [Ins2] and the 5′ end of the sequence segment [Marker2], the sequence segment [TRE]-[1-SceI] or [GRE]-[I-SceI] is inserted in the same or [I-SceI]-[TRE] or [1-SceI]-[GRE] in the reverse orientation, wherein
[I-SceI] is the coding sequence for meganuclease I-SceI from Saccharomyces cerevisiae and
[TRE] is a promoter (TRE) and
[GRE] is a promoter (GRE),
wherein the vector is capable of expressing I-SceI as a function of tetracyclin or estradiol via a promoter (TRE or GRE) which is regulated by a transcription factor responsive to tetracyclin or estradiol.

32. Vector, characterised in that it is derived from a vector according to claims 10 to 30 in which [Ins2] is a transcription factor controlled by tetracyclin or estradiol, by inserting, between the 3′ end of the sequence segment [Ins2] and the 5′ end of the sequence segment [Marker2] the sequence segment [TRE]-[Gen] or [GRE]-[Gen] in the same or [Gen]-[GRE] in the reverse orientation, wherein
[Gen] is the nucleic acid sequence encoding a protein which plays a role for the analysis of repair, genome stability, genotoxicity, or cancer susceptibilities and
[TRE] is a promoter (TRE) and
[GRE] is a promoter (GRE)
wherein the vector is capable of expressing Gen as a function of tetracyclin or estradiol via a promoter (TRE or GRE), which is regulated by a transcription factor responsive to tetracyclin or estradiol.

33. Vector according to claim 31 or 32, characterised in that the transcription factor is the transactivator rtTA reversely controlled by tetracyclin or the GalER-VP activated by estradiol.

34. Vector according to one of claims 1 to 33, characterised in that it is a retroviral vector.

35. Retroviral particles characterised in that they contain a vector according to claims 1 to 34.

36. Eukaryotic cell characterised in that it is transfected with a vector according to claims 1 to 34 or infected with retroviruses according to claim 35.

37. Eukaryotic cell according to claim 36 characterised in that it is a mammalian cell.

38. Transgenic, non-human mammal characterised in that its germ and somatic cells comprise the sequence
5′-[Ins1]-[Prom1]-[Marker1]-[Prom2]-[Ins2]-[Marker2]-3′
in which the sequence segments [Ins1], [Prom1], [Marker1], [Prom2], [Ins2], and [Marker2] have the meaning indicated in claim 1.

39. Mammal according to claim 38, characterised in that the wild-type gene (AFP) is the nucleic acid sequence which codes for the humanised renilla green fluorescent protein (hrGFP), for the enhanced green fluorescent protein (EGFP), for the enhanced blue fluorescent protein (EBFP), for the enhanced cyan fluorescent protein (ECFP), for the enhanced yellow fluorescent protein (EYFP), or for the red fluorescent protein (RFP or DsRed) or a nucleic acid sequence which codes for an enzyme whose activity is detectable via a chemoluminescence reaction.

40. Mammal according to claim 38 or 39 characterised in that [Marker1] and [Marker2] are two random sequence segments homologous, however, over at least 200 bp, wherein a loss or modification of [Ins2] occurs in the case of non-conservative homologous recombination or replication slippage between [Marker1] and [Marker2], inactivation of the gene product concerned occurs in the case of inactivating mutations in [Ins1] or [Ins2], the activation of the gene product concerned occurs in the case of reverse mutations in [Ins1] or [Ins2] and the wild-type gene is reconstituted by homologous DNA exchange if [Marker1] and [Marker2] are sequence segments which code for an autofluorescent protein, for a bioluminescent enzyme or for an enzyme that converts chemiluminescent substrates or for a mutant thereof.

41. Mammal according to claims 38 to 40, characterised in that [Ins1] is a sequence segment which contains a selection marker gene.

42. Mammal according to claims 38 to 40, characterised in that [Ins1] is a sequence segment which contains a nucleic acid sequence which codes for an autofluorescent protein or for a bioluminescent enzyme, or an enzyme that converts chemiluminescent substrates or for a mutant thereof.

43. Mammal according to claims 38 to 42, characterised in that [Ins2] is a sequence segment which contains a selection marker gene.

44. Mammal according to claims 38 to 42, characterised in that [Ins2] is a sequence segment which contains a nucleic acid sequence which codes for an autofluorescent protein, for a bioluminescent enzyme, or for an enzyme that converts chemiluminescent substrates or for a mutant thereof.

45. Mammal according to claims 38 to 44, characterised in that [Ins2] is a sequence segment which contains a nucleic acid sequence which codes for a transcription factor controlled by tetracyclin or estradiol or a nucleic acid sequence coding for I-SceI, a gene to be analysed regarding its possible functions during repair, genome stabilisation, genotoxic effect or the generation of susceptibilities to cancer or a spacer with a preferred length of 1 kb.

46. Mammal according to claims 38 to 45, characterised in that [Prom1] is missing or a CMV promoter or an alternative eukaryotic promoter by means of which an AFP expression is achieved which is just as high as when a CMV promoter is used.

47. Mammal according to claims 38 to 46, characterised in that [Prom2] is missing, a spacer with a length of 1 kb or a promoter from the group consisting of the promoter responsive. to Gal4ER-VP (GREs), the SV40 promoter, the CMV promoter, or a promotor effecting an expression similarly high in various cell and tissue types as the CMV promoter, the latter for AFP genes contained in the sequence segment [Ins2].

48. Mammal according to claims 38 to 47, characterised in that the autofluorescent protein is selected from the group consisting of humanised renilla green fluorescent protein (hrGFP), enhanced green fluorescent protein (EGFP), enhanced blue fluorescent protein (EBFP), enhanced cyan fluorescent protein (ECFP), enhanced yellow fluorescent protein (EYFP), red fluorescent protein (RFP or DsRed) or variants or mutants of these proteins.

49. Mammal according to claims 38 to 47, characterised in that the bioluminescent enzyme or the enzyme that converts chemiluminescent substrates are selected from the group of alkaline phosphatase, β-galactosidase, peroxidase, luciferase, or a variant or mutant thereof.

50. Mammal according to claims 38 to 49, characterised in that [Marker1] and [Marker2] each contain at least one mutation, the positions of the mutations being selected such that
a) the section of the sequence between the position in [Marker2] which corresponds to a mutation in [Marker1] and the first mutation in [Marker2] which is situated in the 3′ direction, or
b) the section of the sequence between the position in [Marker1] which corresponds to a mutation in [Marker2] and the first mutation in [Marker1] which is situated in the 3′ direction
comprises at least approximately 150 base pairs.

51. Mammal according to claims 38 to 50, characterised in that the sequence segment [Prom1] is deleted and the sequence segment [Marker1] immediately starts at the 3′ end of the sequence segment [Ins1].

52. Mammal whose germ and somatic cells contain a transgenic nucleic acid sequence characterised in that the sequence is derived from the sequence in claims 38 to 50 by the sequence segments [[Prom1]-[Marker1]] and [Marker2] being exchanged in their positions, in which the orientation of both sequence segments is simultaneously reversed.

53. Mammal whose germ and somatic cells contain a transgenic nucleic acid sequence characterised in that the sequence is derived from a sequence in claims 38 to 50 by the orientation of the sequence segments [[Prom1]-[Marker1]] and [Marker2] being reversed or exchanged and is the sequence
5′-[Ins1]-(3′-[Marker1]-[Prom1]-5′)-[Prom2]-[Ins2]-[Marker2]-3′,
5′-[Ins1]-[Prom1]-[Marker1]-[Prom2]-[Ins2]-(3′-[Marker2]-5′)-3′,
5′-[Ins1]-(3′-[Marker1]-[Prom1]-5′)-[Prom2]-[Ins2]-(3′-[Marker2]-5′)-3′or
5′-[Ins1]-[Marker2]-[Prom2]-[Ins2]-[Prom1]-[Marker1]-3′

54. Mammal whose germ and somatic cells contain a transgenic nucleic acid sequence characterised in that the sequence is derived from the sequence in claims 38 to 53 by the sequence segment [Marker2] being deleted.

55. Mammal whose germ and somatic cells contain a transgenic nucleic acid sequence characterised in that the sequence is derived from the sequence in claims 38 to 54 in which the vector contains a recognition sequence for meganuclease I-SceI from Saccharomyces.

56. Mammal whose germ and somatic cells contain a transgenic nucleic acid sequence characterised in that the sequence is derived from the sequence in claims 38 to 55 in which [Ins2] is a transcription factor controlled by tetracyclin or estradiol, by inserting, between the 3′ end of the sequence segment [Ins2] and the 5′ end of the sequence segment [Marker2], the sequence segment [TRE]-[I-SceI] or [GRE]-[I-SceI] in the same or [1-SceI]-[TRE] or [I-SceI]-[GRE] in the reverse orientation, wherein
[I-SceI] is the coding sequence for the meganuclease I-SceI from Saccharomyces cerevisiae and
[TRE] is a promoter (TRE) and
[GRE] is a promoter (GRE),
wherein I-SceI is expressed in the presence of tetracyclin or estradiol via a promoter (TRE) or (GRE), which is regulated as a function of the transcription factor controlled by tetracyclin or estradiol.

57. Mammal whose germ and somatic cells contain a transgenic nucleic acid sequence characterised in that the sequence is derived from the sequence in claims 38 to 55 in which [Ins2] is a transcription factor controlled by tetracyclin or estradiol, by inserting, between the 3′ end of the sequence segment [Ins2] and the 5′ end of the sequence segment [Marker2], the sequence segment [TRE]-[Gen] or [GRE]-[Gen] in the same or [Gen]-[GRE] in the reverse orientation, wherein
[Gen] is the nucleic acid sequence encoding a protein which plays a role for the analysis of repair, genome stability, genotoxicity, or cancer susceptibilities and
[TRE] is a promoter (TRE) and
[GRE] is a promoter (GRE),
wherein the vector is capable of expressing Gen as a function of tetracyclin or estradiol via a promoter (TRE or GRE), which is regulated by a transcription factor responsive to tetracyclin or estradiol.

58. Mammal according to claims 56 or 57 characterised in that the transcription factor is the transactivator rtTA reversely controlled by tetracyclin or the GalER-VP activated by estradiol.

59. Mammal according to claims 38 to 58, characterised in that it is a rodent.

60. Process for the determination of genotoxicities characterised in that cells according to claims 36 or 37 or mammals according to claims 38 to 59 are brought into contact with a test compound, wherein the appearance, increase, or decrease in fluorescent or luminescent cells corresponding to the activity of the wild-type gene product of [Marker1], [Marker2], [Ins1], or [Ins2] in the above-mentioned cells or in the cells of the above-mentioned mammal, subsequently measured by FACS analysis, fluorescence measurement, fluorescence microscopy, laser scanning cytometry, or by a luminescence detection reaction, indicates the genotoxic effect of the test compound.

61. Process for the detection of the susceptibility of an eukaryotic individual for the appearance or progression of cancer characterised in that an analysis of the patient's blood, skin, or biopsy material is carried out during which the vector according to claim 1 is introduced into the cells, the DNA exchange frequencies being analysed by FACS analysis, fluorescence microscopy, laser scanning cytometry, fluorescence measurement, or luminescence detection reaction on transfected cells.

62. Process for the analysis of conservative homologous recombination processes, non-conservative homologous recombination processes, end joining, inactivating and reverse mutations separately or in the same batch, characterised in that cells according to claim 36 or 37 or mammals according to claims 38 to 59 are brought into contact with a test compound, transfected with a I-SceI expression plasmid, I-SceI is expressed by induction or left untreated, DNA exchange frequencies being subsequently determined by FACS analysis, fluorescence measurement, fluorescent microscopy, laser scanning cytometry, or by a luminescence detection reaction, these events being indicated by fluorescent or luminescent cells corresponding to the activity of the wild-type gene product of [Marker1] and [Marker2],
wherein the loss of a fluorescence or luminescence activity in individual cells of the population indicates non-conservative DNA exchange events, replication slippage, or inactivating mutations if [Ins2] of the vector used is the coding sequence of the AFP or LE and if [Marker1] and [Marker2] are homologous sequences of at least approximately 200 base pairs,
wherein, in as far as [Ins1] and [Ins2] represent AFP or LE genes inactivated by mutation, reverse mutations are detected in [Ins2] or in [Ins1] by the appearance of a fluorescence or luminescence signal of the products of the wild-type gene concerned.

63. Process for characterising the meaning of a gene with respect to its possible functions in maintaining or reducing genetic stability characterised in that the process is carried out according to claim 62, cells or mammals with different status being used as regards the gene to be investigated.

64. Process according to claim 63 characterised in that the coding sequence of the gene to be investigated or a mutated form thereof is introduced into the cells or mammals as [Ins1] or [Ins2] within the same vector or on a separate expression vector via transfection or retroviral infection, in which recombination, end joining, replication slippage, or mutations are compared with respect to a vector with or without the coding sequence, a vector with wild-type or mutant sequence, or the same vector following gene activation via promoter induction, e.g. by estradiol administration, in which the proportion of fluorescent or luminescent cells indicates the frequencies with which DNA exchange events or mutations have taken place.

65. Process for the determination of the genetic stability or instability of a cell type or the recombination or mutation frequencies of a cell type, tissue type or an eukaryotic organism characterised in that the process is carried out according to claim 62, in which cells, tissues, or organisms of different types are used into which the vectors according to claims 1 to 34 are introduced so that cells according to claims 36 or 37 or mammals according to claims 38 to 59 are generated, wherein subsequently appearing fluorescences are compared with those of control cells, control tissues, or control organisms which do not contain these vectors, wherein a cell type, tissue type of the organism is classified as genetically unstable, if it exhibits significantly increased DNA exchange or mutation rates compared with the controls.

66. Kit for carrying out the process according to claim 60 using cells according to claims 36 or 37, characterised in that it contains cells which are transfected with a vector according to claims 1 to 34 or retrovirally infected.

67. Kit for carrying out the process according to claims 61 to 65, characterised in that it contains cells which are transfected with a vector according to claims 1 to 34 or retrovirally infected.

68. Kit for carrying out the process according to claims 60 to 65, characterised in that it contains vectors according to 1 to 34, retroviral particles according to claim 35.
